(19) United States
(12) Patent Application Publication
(10) Pub. No.: US 2003/0113330 A1
(43) Pub. Date: Jun. 19, 2003

(54) Methods for treating pulmonary fibrosis

(76) Inventor: Bruce D. Uhal, East Lansing, MI (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/337,169

(22) Filed: Jan. 6, 2003

Related U.S. Application Data
(62) Division of application No. 09/708,742, filed on Nov. 8, 2000.
(60) Provisional application No. 60/164,052, filed on Nov. 8, 1999.

(51) Int. Cl. A61K 39/395; A61K 31/56; A61K 31/4178

(52) U.S. Cl. 424/146.1; 514/177; 514/381

(57) ABSTRACT

The present invention relates to uses of non-thiol ACE inhibitors, angiotensin II antagonists, angiotensin II receptor antagonists, endonuclease inhibitors, and caspase-inhibitors to treat pulmonary fibrosis and/or inhibit pulmonary epithelial cell apoptosis, including pulmonary fibrosis associated with amiodarone product toxicity.

### RELATED APPLICATIONS

This application claims priority of U.S. Serial No. 60/164,052 filed Nov. 8, 1999, the entire disclosure of which is incorporated by reference herein.

### STATEMENT AS TO FEDERALLY SPONSORED RESEARCH

This invention was supported in part by the U.S. Government under PHS HL-45136. The Government has certain rights in the invention.

### FIELD OF THE INVENTION

The present invention relates generally to novel therapeutic methods for treating pulmonary fibrosis using compounds that inhibit apoptosis of cells mediated by the renin-angiotensin-aldosterone system.

### BACKGROUND OF THE INVENTION

Intact alveolar epithelium is critical in maintaining many lung functions including gas exchange, water balance, surfactant synthesis and local immunomodulation. Damage to the human lung can result from either blood-borne or inhaled toxicants or by inflammation of extrapulmonary origin, such as that associated with sepsis, although injury by inhalation is the most common mechanism. Studies have documented and detailed the lung injuries induced by a variety of mineral dusts, see, e.g., Miller et al., Environ Health Perspect. 85:15-23 (1990), oxidant gases, see, e.g., Evans et al., Arch. Environ. Health 24:180-188 (1972), and cytotoxic drugs, see, e.g., Thrall et al., Am. J. Pathol. 95:117-130 (1979).

Approximately two-thirds of all cases of interstitial lung disease (ILD) are of unknown etiology. It has been estimated that the ILD of unknown etiology are responsible for more than 10,000 hospital admissions in the United States alone per year and have a prevalence of approximately 5 to 10 cases per 100,000 population. The most common ILD of unknown etiology are idiopathic pulmonary fibrosis, sarcoidosis, and chronic ILD associated with the collagen-vascular disorders. Less common syndromes are histiocytosis-X, Goodpasture's syndrome, chronic eosinophilic pneumonia, idiopathic pulmonary hemosiderosis, and the ILD associated with the pulmonary vasculitides.

For approximately one-third of patients with ILD a specific etiological agent can be identified. These diseases are most commonly associated with the inhalation of organic or inorganic dusts, particularly crystalline silica and silicates such as asbestosis, the hypersensitivity pneumonitides, and drug-induced ILD. Less frequent are the diseases associated with exposure to paraquat, radiation, infectious agents, and the inhalation of noxious gases, aerosols, chemical dusts, fumes or vapors. See generally Crystal, Interstitial Lung Disease in Cecil Textbook of Medicine (17th Ed.) (Wyngaarden and Smith, Eds.), W. B. Saunders Co., Philadelphia, Pa. (1985) at p. 406.

Although standard therapeutic regimens are in use for the management of fibrotic lung disorders, these regimens have achieved only very limited success. Traditionally, corticosteroids in relatively high doses have been most widely used for the treatment of this class of lung diseases. However, most patients also concurrently receive an additional pharmacologic agent of another type which may itself be cytotoxic, such as cyclophosphamide, chlorambucil, or cyclosporine. See Hunninghake et al., Am. J. Resp. Crit. Care Med. 151:915-918 (1995). Although only a minor percentage of patients (20%) that receive the second agent respond positively to it, those who do have a better prognosis than patients who receive corticosteroids alone. See id. The rationale of the traditional therapies has relied on the anti-inflammatory action of the corticosteroids in conjunction with the second agent's “antifibrotic” potential, which often was merely inferred from its ability to inhibit lung fibrogenesis in an animal model, with little specific information regarding the underlying mechanisms. Unfortunately, the side effects of the corticosteroids, which include alterations in fluid and electrolyte balance, altered immune response, cataracts, myopathy, osteoporosis and behavioral changes, are serious and numerous. There exists a need for new antifibrotic therapies that could reduce pulmonologists' reliance on corticosteroids. See, e.g., Raghu, Mayo Clin. Proc. 72(3):285-7 (1997).

In general, the alveolar epithelial type I cell is widely accepted to be particularly sensitive to damage by any cytotoxic agent for two reasons: (a) the type I cell, because it covers 95% of the surface area of the very thin gas exchange structures, is very flat and extremely thin, and is thus exposed to inhaled substances to a greater degree than other cells, and (b) unlike its neighbor and progenitor the type II cell, the type I cell is relatively devoid of inducible antioxidant defense mechanisms such as catalase and superoxide dismutase (SOD).

Studies such as those reviewed in Uhal, Am. J. Physiol. 272:L1031-L1045 (1997), documented the role of the neighboring type II cell in replacing damaged or lost type I cells through regulated cell division and terminal differentiation. Tangible evidence of this concept was provided by the findings that lung injuries in which damage is limited to the type I cell are generally reparable, but injuries that kill both the type I and the parent type II cell (such as that caused by Paraquat) are fatal because they eliminate the only stem cell that can replace the type I “barrier lining” cells. Much of the evidence in support of this concept was obtained by histologic studies in which cell injury or death was scored by classical definitions of necrosis or cell loss, which are prominent in the alveolar epithelia of animal or human lungs injured by the agents described above. Repair of the alveolar epithelium is accomplished by the regulated proliferation and differentiation of type II alveolar epithelial cells, which replace both type II and type I alveolar epithelial cells lost to injury or normal turnover. See Uhal, Am. J. Physiol. 272:L1031-L1045 (1997). For these reasons, the type II alveolar epithelial cell is believed to be a critical player in the pathogenesis of pulmonary fibrosis. See Simon, in Pulmonary Fibrosis, Phan and Thrall (eds.) in Lung Biology in Health and Disease, C. Lenfant (exec. ed.) 80:511-540 (1995). In both humans and animal models, fibrosis in the lung decreases gas exchange due to thickened alveolar septa, decreases lung elasticity, and thus makes breathing difficult. Eventually this causes death, as the blood gases become abnormal, lung fluid clearance becomes inefficient, and breathing becomes prohibitively labored.

Efficient repair of damage to the alveolar epithelium is believed to be essential for healing of such injuries without ongoing fibrosis, and some authors have contended that damage to the alveolar epithelium appears to be required for the induction of lung fibrogenesis. See, e.g., Hunninghake, et al., Am. J. Resp. Crit. Care Med. 151:915-918 (1995). Investigations of lung injury in animal models have suggested that incomplete or delayed alveolar repair leads to acceleration of collagen deposition and lung fibroblast proliferation. See Adamson, et al., Am. J. Pathol. 130:377-383 (1988); Haschek et al., Toxicol. Appl. Pharmacol. 51:475-487 (1979). A relationship between incomplete epithelial repair and fibrogenesis within the underlying interstitium has also been postulated in biopsy specimens from patients with fibrotic lung disease. See Kuhn III et al., Am. Rev. Resp. Dis. 140:1693-1703 (1989). In addition to providing intact barrier functions, see Chaucey et al., Lab Invest. 58:133-140 (1988), the alveolar epithelium is believed to be an important constitutive producer of prostaglandin E2 (PGE2), an inhibitor of fibroblast proliferation. See Barile et al., Arch. Biochem. Biophys. 265:441-446 (1988). Normal alveolar epithelial cells also produce urokinase-type plasminogen activator (uPA) and PA inhibitor-1, and exhibit potent capacity for the degradation of fibrin clots in vitro. See Simon et al., Am. J. Physiol. 262:L482-488 (1992).

On the other hand, a number of profibrotic sequelae of denudation of the alveolar epithelium have been proposed. Alveolar epithelial cells express urokinase-type plasminogen activator and plasminogen activator inhibitor-1 and are capable of degrading fibrin clots in vitro, and this capacity is believed to act in a tonic fashion to clear intra-alveolar fibrin in vivo. See Simon et al., Am. J. Physiol. 262:L482-488 (1992). In addition, AEC are known to produce E-series prostaglandins, which are documented inhibitors of lung fibroblast proliferation, see Chaucey et al., Lab Invest., 58:133-140 (1988) and Barile et al., Arch. Biochem. Biophys. 265:441-446 (1988), and AEC provide a physical barrier necessary for transport functions and to protect underlying interstitial cells from the cytokines released by free alveolar phagocytes. For these reasons, the loss of AEC and/or interruption of their replacement by chronic apoptosis could be envisioned as permissive for fibroblast proliferation and activation by macrophage-derived factors and for the accumulation of intra-alveolar fibrin. Other potential antifibrotic functions of the alveolar epithelium were discussed in Simon, Lung Biology in Health and Disease 80:511-540 (1995).

The widespread use of amiodarone (AM) for the treatment of ventricular and supraventricular cardiac arrhythmias has led to increased interest in its side effects, particularly pulmonary toxicity. Amiodarone (AM) is a benzofuran derivative with class III antiarrhythmic activity that is effective in controlling intractable cardiac arrhythmias (see Herre et al., J. Am. Coll. Cardiol. 13: 442-9,(1989) and Singh, S. N. et al. N. Engl. J. Med. 333: 77-82, (1995). Evidence suggests that the drug may have a role in reducing the relative risk for arrythmic or sudden death and overall mortality in survivors of myocardial infarction (Ceremuzynski et al. J. Am. Coll. Cardiol. 20: 1056-62, (1992) and Pfisterer et al. Am. J. Cardiol. 69:1399-1402, (1992), and in heart failure patients (see Dovac et al. Lancet 344:493-498, 1994 and Janse, M., et al. Eur. Heart J. 19:85-95, 1998). Its clinical use, however, is limited by amiodarone-induced pulmonary toxicity (AIPT), which occurs with an incidence of 6% and an estimated mortality of 5 to 10% of affected patients (see Pfisterer, M., et al. Am. J. Cardiol. 69:1399-1402, 1992). Moreover, it is now known that pulmonary toxicity is the most significant, most limiting and potentially life-threatening side effect associated with AM use (see Marchlinski, J. L., et al., Ann. Intern. Med 97:939-945, 1982 and Martin, W. J. II, et al. Chest 93: 1067-1075, 1998 and Pitcher, W. D. Am. J. Med. Sci. 303: 206-212, 1992).

The pathology of AIPT, which includes alveolitis, phospholipidosis and in its advanced stages, irreversible fibrosis, is well-described but the underlying mechanisms remain unclear (see Reasor, M. J. and S. Kacew. Proc. Soc. Exp. Biol. Med. 212: 297-304, 1996). A number of theoretical pathogenic mechanisms are suggested by animal and cell culture studies, including alterations in membrane properties, increases in intracellular free calcium (Pitcher, W. D. Am. J. Med. Sci. 303: 206-212, 1992), generation of radicals (see Vereckei, A., et al., J. Cardiovasc. Electrophysiol. 4: 161-177, 1993. J. Am. Coll. Cardiol. 30(2):514-7,1997), lung phospholipidosis (see Reasor, M. J., et al., Am. Rev. Pespir. Dis 137:510-518, 1988), immunologic mechanisms (1) and direct cytotoxicity (see Reasor, M. J. and S. Kacew. Proc. Soc. Exp. Biol. Med. 212: 297-304, 1996). Although the latter potential mechanism has been studied in isolated lung fibroblasts, endothelial cells and alveolar macrophages (see Reasor, M. J. and S. Kacew. Exp. Biol. Med. 212: 297-304, 1996), and it has been shown previously that AM and its active metabolite desethylamiodarone (DES) are directly cytotoxic to cell types other than alveolar epithelial cells, such as bovine arterial endothelial cells (Martin, W. J. II, et al., Am. J. Pathol. 120:344-352, 1985), alveolar macrophages (see Martin, W. J. II, et al., supra and Ogle, C. L., et al., Toxicology 62(2): 227-38, 1990), human pulmonary artery endothelial cells (see Powis, G., et al., Toxicol. Appi. Pharmacol 103: 156-164, 1990), interstitial lung fibroblasts (see Martin, W. J. II, et al., supra), bronchial epithelial cells (Colgan, T., et al., Ultrastruct. Pathol 6:199-204, 1984) and hepatocytes (see Gross, S. A., et al., Proc. Soc. Exp. Biol. Med. 190:163-170, 1983), no studies to date have explored the possibility that AM or DES might be capable of inducing apoptosis.

Several mechanisms underlying the adverse pulmonary effects of amiodarone have been proposed. In addition to the direct cellular damage referred to in the preceding paragraph, these include derarrangements in lipid metabolism associated with the induction of phospholipidosis (Camus, P., et al., Radiology 150:279-280, 1984), immune-mediated mechanisms such as the activation of natural killer cell activity (Karpel, J. P., et al., Chest 99:230-234, 1991) and the development of edema associated with an increased production of superoxide anion (Kennedy, T. P., et al., J. Cardiovasc. Pharmacol. 12:23-36, 1988). The tissue level of reduced glutathione was also found to be increased significantly. In contrast, pretreatment with antioxidant agents such as butylated hydroxyanisole, vitamin E, N-acetylcysteine or ventilation with 40% oxygen protected against amiodarone-induced edema (see Kennedy, T. P. et al., supra). However, the relationship of these proposed mechanisms to the induction of lung fibrogenesis is unclear.

Apoptosis is a cell death pathway that is highly conserved throughout evolution. See Ameisen, et al., Science 272:1278-1279 (1996). Apoptosis is characterized by membrane blebbing and loss of integrity, cellular and cytoplasmic shrinkage, chromosomal fragmentation and condensation, and endonuclease activation resulting in a characteristic 180 basepair (bp) DNA ladder. See Yang et al., Blood 88:386-401 (1996). A number of in vitro and in vivo stresses can induce apoptosis, including glucocorticoid administration, hormonal deficiencies, chemotherapy, mechanical injury, and DNA damage. Apoptosis is also induced by aberrant cell cycle activity, and can be triggered in cells that express the FAS receptor following activation of the FAS receptor by its natural binding partner, the FAS ligand. Cells expressing the FAS ligand bind to cells that express the FAS receptor and thereby initiate a cascade that can result in apoptosis. See Nagata, et al., Science 267:1449-1456 (1995).

Recent investigations suggest an important role for apoptosis of AEC in animal models of lung fibrosis (Hagimoto, N., et al., Am. J. Respir. Cell Mol. Biol. 17:272-278, 1997) and in the advanced stages of fibrotic lung disease in humans (see Bardales, R. H., et al., Am. J. Pathol 149: 845-852, 1996 and Uhal, B. D., et al., Am. J. Physiol. 275:L1192-L1199, 1998). For example, apoptosis is a major pathway responsible for the resolution of type II pneumocytes in acute lung injury. Am. J. Pathol 149: 845-852, 1996 and Uhal, B. D., et al., Am. J. Physiol. 275:L1192-L1199, 1998). Apoptosis has been shown to be an important component of the elimination of excess mesenchymal cells from diseased or injured human lung. See, e.g., Polunovsky, et al., J. Clin. Invest. 92:388-397 (1993). Apoptosis is also thought to be involved in the removal of excess epithelial cells in remodeling hyperplastic human lung. See Bardales et al., Am. J. Pathol. 149:845-852 (1996). Apoptosis is also a prominent feature of the fibrotic lung in vivo. See Bardales, et al., Am. J. Pathol. 149:845-852 (1996). See also Hagimoto et al., Am. J. Respir. Cell Mol. Biol. 16:91-101 (1997), and Kuwano et al., Am. J. Resp. Crit. Care Med. 154:477-483 (1996). On the other hand, apoptosis and increased expression of the FAS protein (also known as APO1 or CD95) were observed in both bronchial and alveolar epithelial cells during the pathogenesis of bleomycin-induced pulmonary fibrosis in mice. See Hagimoto, et al., Am. J. Respir. Cell Mol. Biol. 16:91-101 (1997). Recently, ligation of FAS antigens and receptors in vivo, by intratracheal administration of anti-FAS antibodies, was shown to induce epithelial cell apoptosis, and pulmonary fibrosis in mice. See Hagimoto, et al., Am. J. Respir. Cell Mol. Biol. 17:272-278 (1997). Apoptosis in the lung, and more specifically apoptosis of alveolar epithelial cells, is a phenomenon only very recently discovered. Very little is yet known about the regulation of this phenomenon, and even less is known about its pharmacologic manipulation.

The renin-angiotensin-aldosterone system, which plays a role in the regulation of blood pressure and blood volume, has not hitherto been demonstrated to play a role in apoptosis of alveolar epithelial cells. Renin hydrolyzes angiotensinogen, a serum globulin secreted by the liver, to produce the decapeptide angiotensin I. Angiotensin I converted by the peptidase angiotensin converting enzyme (ACE), which is present chiefly in the lungs, to form the octapeptide hormone angiotensin II, a powerful vasopressor that not only raises blood pressure but also directly stimulates secretion of aldosterone by the adrenal cortex. Angiotensin II is also further hydrolyzed to form the heptapeptide angiotensin m, which has lesser vasopressor activity but is more active on the adrenal cortex. Aldosterone regulates electrolyte and water balance by promoting the retention of sodium (and therefore the retention of water) and the excretion of potassium. The retention of water causes an increase in plasma volume and blood pressure.

Angiotensin converting enzyme (ACE) is an enzyme which catalyzes the conversion of angiotensin I to angiotensin II. ACE inhibitors have been used medically to treat hypertension, congestive heart failure, myocardial infarction and renal disease. Captopril, a thiol ACE inhibitor, has been shown to inhibit fibrogenesis in the lung, but the mechanisms underlying this action remain unclear. For example, captopril has been shown to ameliorate radiation-induced fibrosis of the lung (Ward et al., Int. J. Radiat. Oncol. Biol. Phys. 19:1405-1409 (1990)) as well as lung fibrosis induced by the plant alkaloid monocrotaline (Molteni et al., Proc. Soc. Exp. Biol. Med. 180:112-120 (1985)). Captopril has also been shown to ameliorate radiation-induced fibrosis of the kidney. See Cohen et al., Lab. Invest. 75:349-360 (1996).

Examinations of human lung fibroblasts in culture suggest that the ameliorative action of captopril might be due to direct inhibitory effects on fibroblast proliferation, which were observed in the presence of the mesenchymal cell mitogen basic FGF (bFGF). See Nguyen et al., Proc. Soc. Exp. Biol. Med. 205:80-84 (1994). Captopril is also known to inhibit the proliferation of cultured human mammary duct carcinoma cells, (Small et al., Breast Canc. Res. Treat. 44:217-224 (1997)), and to slow the growth rate of experimental fibrosarcomas in rats (Volpert et al., J. Clin. Invest. 98:671-679 (1996). Direct inhibition by captopril of the zinc-dependent 72 kD and 92 kD metalloproteinases produced by endothelial cells has also been reported.

The angiotensin II receptor has been well-studied at least in regard to its roles in the regulation of vascular tone. The receptor exists as two major subtypes termed AT1 (the type 1 receptor) and AT2 (the type 2 receptor) that are expressed individually or together in a wide variety of cell types. See Timmermans, et al., Pharmacol. Reviews 45:205-251 (1993). The AT1 receptor functions through the now-classical pathway of phospholipase A2 activation, phosphatidyl inositol generation and release of intracellular calcium, whereas the AT2 receptor is believed to function through the involvement of tyrosine phosphatases. Angiotensin II has been shown to induce apoptosis through binding to at least one of these receptors on cardiac myocytes (Kajstura et al., J. Mol. Cell. Cardiol. 29:859-870 (1997)), and on endothelial cells (Dimmeler et al., Circ. Res. 81:970-976 (1997)). Although in vitro experiments suggest that the AT2 receptor is involved in apoptosis of mouse fibroblast and rat pheochromocytoma cell lines (Yamada et al., Proc. Nat. Acad. Sci. 93:156-160 (1996)), pharmacologic studies have implicated the AT1 receptor in apoptosis of rat ventricular myocytes (Kajstura, et al., J. Mol. Cell. Cardiol. 29:859-870 (1997)).

Several peptide antagonists of the angiotensin II type 1 and/or 2 receptors are available. Saralasin, a substituted octapeptide analog of angiotensin II, is the prototypical synthetic peptide antagonist of the angiotensin II receptor. See Regoli et al., Pharmacol. Rev. 26:69-123 (1974). Saralasin blocks the binding of angiotensin II to either of its receptor subtypes AT1 or AT2, in contrast to losartan or compound L158809, which are selective for receptor subtype AT1, or PD126055, which is selective tor AT2. Although the peptide antagonists were among the first to be synthesized in the hope of creating new therapies for hypertension, they were found to be of limited clinical value for treating hypertension because of low oral bioavailability and partial agonist activity. See Timmermans et al., Pharmacol. Reviews 45:205-251 (1993).

In contrast, losartan (DuP 753) is a very effective and orally active non-peptide AT1 receptor antagonist that was approved for use in the United States in 1995. Recent in vitro studies indicate that losartan also is effective in blocking apoptosis in cardiac myocytes in response to several stimuli. See Leri et al., J. Clin. Invest. 101:1326-42 (1998), and Kajstura et al., J. Mol. Cell. Cardiol. 29:859-870 (1997). The structurally related analog L158809 is also a very potent AT1 antagonist. See Timmermans et al., Pharmacol. Reviews 45:205-251 (1993). L158809 was recently shown to have anti-fibrotic properties in an experimental model of total bone marrow transplant (see Molteni et al., 10th International Colloquium on Lung Fibrosis, Siena, Italy (Oct. 14, 1998).

The caspase cysteine proteases are a family of regulatory enzymes required for the execution of apoptosis in all cell types examined thus far. See Schwartz Circulation 97:227-229 (1998). To date, the only published information regarding the specific roles of caspases in apoptosis of alveolar epithelial cells is from Wen et al., Am. J. Physiol. 273:L921-L929 (1997). Using human A549 cells, Wen and coworkers showed that exposure to interferon-c (INF-c) induced an increase in the abundance of immunoreactive interleukin-1b converting enzyme (ICE, Caspase 1), whereas exposure to an anti-Fas antibody alone did not. See Yonehara, et al., J. Exp. Med. 169:1747-1756 (1989); Kobayashi, et al., Proc. Natl. Acad. Sci. 87:9620-9624 (1990); and Itoh, et al., Cell 66:233-243 (1991).

In view of the foregoing, a need exists in the art to better understand the cellular and molecular processes underlying pulmonary epithelial cell apoptosis and pulmonary fibrosis, and to develop improved drug therapies to replace or supplement the existing methods for treating these conditions. Preferably, these drug therapies would be designed to reduce or prevent these conditions at their earliest stages, but could also be useful during the latter stages of such conditions, when patients become symptomatic.

### SUMMARY OF THE INVENTION

The present invention provides novel therapeutic uses for compounds which interact with the renin-angiotensin-aldosterone system (including any one of the following: thiol and non-thiol ACE inhibitors, angiotensin II antagonists, angiotensin II receptor antagonists, aldosterone antagonists, caspase enzyme inhibitors and endonuclease inhibitors) and/or TNF receptor system and/or FAS receptor/ligand system in subjects at risk of or suffering from pulmonary epithelial cell apoptosis and/or pulmonary fibrosis and conditions associated therewith.

Thus, according to one aspect of the invention, methods are provided for treating pulmonary fibrotic disease by administering to a subject at risk of or suffering from a pulmonary fibrotic disease a pharmaceutically effective amount of an antagonist of a member of the renin-angiotensin-aldosterone system (wherein the antagonist may be other than a thiol or non-thiol ACE inhibitor). As used herein “treatment” means prophylactic or therapeutic treatment. An exemplary “pharmaceutically effective amount” is an amount effective to inhibit apoptosis of pulmonary epithelial cells.

According to another aspect of the invention, methods are provided for treating a pulmonary fibrotic disease comprising administering to a subject at risk of or suffering from a pulmonary fibrotic disease a pharmaceutically effective amount of a caspase enzyme inhibitor or an endonuclease inhibitor or a protein kinase C inhibitor.

Specifically contemplated are methods for inhibiting pulmonary epithelial cell apoptosis by administration of an effective amount of a non-thiol ACE inhibitor or other antagonist of the renin-angiotensin-aldosterone system.

According to yet another aspect of the invention, screening methods are provided for identifying novel therapeutic compounds for treating pulmonary fibrotic disease which include steps of: (a) measuring apoptosis of mammalian pulmonary epithelial cells in the presence and absence of a test compound; (b) selecting a test compound that inhibits apoptosis of said cells; and (c) determining an effect of said test compound selected in step (b) in an animal model of pulmonary fibrosis.

Apoptosis of mammalian pulmonary epithelial cells can be measured through, e.g., in situ end-labeling of fragmented DNA, nuclear and chromatin morphological analysis, Caspase-1 or Caspase-3 activity analysis, analysis of binding of Annexin-V, or any other assays described herein or known in the art. Suitable animal models include models in which pulmonary fibrosis is induced by toxic agents such as bleomycin (as described herein) or monocrotaline [Molteni et al., Proc. Soc. Exp. Biol. Med. 180:112-120 (1985)], by radiation [Ward et al., Int. J. Radiat. Oncol. Biol. Phys. 19:1405-1409 (1990)], by Fas-induced apoptosis [Hagimoto, et al., Am. J. Respir. Cell Mol. Biol. 17:272-278 (1997)], or any other models known in the art.

Novel therapeutic compounds for treating pulmonary fibrotic disease can also be identified by screening methods which include steps of: (a) measuring the interaction of a test compound with a member of the renin-angiotensin-aldosterone system (wherein said member may be other than ACE); (b) selecting a test compound that interacts with said member of the renin-angiotensin-aldosterone system; and (c) determining effect of the test compound selected in step (b) in an animal model of pulmonary fibrosis. For example, in step (a), binding of a test compound to angiotensin II or an angiotensin II receptor such as AT1 is measured using any assay known in the art.

Alternatively, novel therapeutic compounds for treating pulmonary fibrotic disease can be identified by screening methods including steps of: (a) measuring the activity of angiotensin converting enzyme (ACE) in the presence and absence of a test compound; (b) selecting a test compound that inhibits ACE; and (c) measuring apoptosis of mammalian pulmonary epithelial cells in the presence and absence of said test compound selected in step (b).

Novel compounds identified by these screening methods are included in the present invention, as are therapeutic uses of such compounds for treating pulmonary fibrotic diseases.

Administration of the therapeutic compounds of the invention to subjects at risk of pulmonary fibrosis or subjects suffering from pulmonary fibrosis is contemplated. Suitable subjects include mammals, particularly humans, or other animals. These therapeutic compounds can be administered at doses ranging from about 1 μg/kg to 100 mg/kg daily, varying in children and adults. The dosage may be administered once daily, or in equivalent doses at longer or shorter intervals, e.g. for 5 days. Presently preferred therapeutic compounds include lisinopril, saralasin, losartan, L158809, irbesartan, ZVAD-cmk or -fmk, DEVD-cmk or -fmk, YVAD-cmk or -fmk, aurintricarboxylic acid or chelerythrine.

The invention further provides use of the therapeutic compounds of the invention in the manufacture of a medicament for preventing or treating pulmonary epithelial cell apoptosis and/or pulmonary fibrosis.

According to another aspect of the invention, methods are provided for administering to subjects in need thereof (e.g., suffering from arrhythmia) an amount of an amiodarone product that is higher than the currently recommended amiodarone-equivalent dose of about 1600 mg/day (or 23 mg/kg/day) or about 1200 mg/day by administering, e.g., concurrently, an antagonist of the renin-angiotensin system or another apoptosis inhibitor. Preferably the amount is higher than about 10, 15, 20, 22.5, 25, 27.5, 30, 32.5, 35, 37.5, or 40 mg/kg/day. Also according to this aspect of the invention, methods are provided for administering to subjects in need thereof a dose or (multiple doses over a long time period, e.g., longer than 1, 2 or 3 weeks, or 1, 2, 3, 4, 5 or 6 months or 1 year) of an amiodarone product that maintains a amiodarone-equivalent mean serum concentration of greater than about 1.8 μg/mL [or mg/L] (e.g., after 48 or 72 hours of i.v. or p.o. treatment), or a mean serum concentration greater than about 1.4, 1.5, 1.6, 1.7, 1.8, 1.9, 2.0, 2.25, 2.5, 2.75, 3.0, 3.25, 3.5, 3.75, 4.0, 4.25, 4.5, 4.75 or 5 μg/mL [or mg/L].

According to yet another aspect of the invention, methods are provided for preventing, reducing or reversing adverse effects (particularly pulmonary toxicity) resulting from administration of an amiodarone product by administering an antagonist of the renin-angiotensin system or another apoptosis inhibitor at a dosage effective to reduce the apoptotic effects of the amiodarone product. Administration of the renin-angiotensin system inhibitor or apoptosis inhibitor can be but need not be concurrent.

Amiodarone products include amiodarone, amiodarone analogs, e.g., dronedarone or amiodarone metabolites, e.g. N-desethylamiodarone (DES). Preferably the amiodarone product is an analog or metabolite of amiodarone, rather than amiodarone itself.

Antagonists of the renin-angiotensin system include AT1 receptor antagonists such as saralasin, or preferably losartan, L158809 or irbesartan (which selectively antagonize AT1); or ACE inhibitors, such as lisinopril, captopril or enalapril. Apoptosis inhibitors include intracellular inhibitors like the caspase inhibitors.

While some patients may have been prescribed the usual recommended doses of amiodarone together with an AT1 inhibitor such as losartan or an ACE inhibitor such as captopril for unrelated reasons (e.g. for antihypertensive treatment), it was not previously known that these second agents reduced pulmonary toxicity of amiodarone.

These and other aspects of the invention will be described in greater detail below. Throughout this disclosure, all technical and scientific terms have the same meaning as commonly understood by one of ordinary skill in the art to which this invention pertains unless defined otherwise. Numerous additional aspects and advantages of the invention will become apparent to those skilled in the art upon consideration of the following detailed description of the invention which describes presently preferred embodiments thereof.

### DETAILED DESCRIPTION OF THE INVENTION

The present invention provides novel therapeutic methods involving the administration of antagonists of the renin-angiotensin-aldosterone system or the FAS receptor/ligand system, i.e., the therapeutic compound(s), for preventing or slowing the progression of pulmonary fibrosis. Unlike conventional therapies, which rely on administration of corticosteroids to combat inflammation, often in combination with other putatively antifibrotic agents that are usually cytotoxic, therapeutic administration of the therapeutic compounds(s) is expected to be effective for preventing and/or treating the underlying disease itself.

The invention also provides methods of preventing, reducing or reversing the adverse effects of amiodarone product administration, particularly the pulmonary toxicity. Such methods allow administration of amiodarone product at a dose that would otherwise result in toxicity or at a dose higher than presently recommended, or to maintain a serum concentration at a level that would otherwise result in toxicity or a level higher than presently recommended.

As used herein, “amiodarone product” includes amiodarone, amiodarone derivatives, analogs and metabolites. When dosages or concentrations of amiodarone product are recited, they are meant to refer to a dose or concentration of the amiodarone analog or metabolite or derivative that provides a therapeutic effect equivalent to that recited dose of amiodarone. Thus, references herein to an “amiodarone-equivalent dose” of 1600 mg/day mean that the dose of amiodarone product (e.g. an analog) provides a therapeutic effect equivalent to 1600 mg/day of amiodarone. Similarly, references herein to maintaining an “amiodarone-equivalent mean serum concentration” of 1.8 μg/mL mean that the mean serum concentration of the amiodarone product or active metabolite thereof provides a therapeutic effect equivalent to a mean serum concentration of 1.8 μg/mL of amiodarone. Exemplary amiodarone products include amiodarone, its active metabolite desethylamiodarone, its analog dronedarone, and other active benzofuran derivatives. Amiodarone products include those compounds disclosed in Sanofi patents U.S. Pat. No. 5,266,711, WO 98/58643, WO 98/40067, and WO 97/02031, the complete disclosures of all of which are incorporated herein by reference.

Antagonists of the renin-angiotensin-aldosterone system include any one of the following: thiol or non-thiol ACE inhibitors, angiotensin II antagonists, angiotensin II receptor antagonists, and aldosterone antagonists. Such antagonists also specifically include antibodies to components of the renin-angiotensin-aldosterone system or antisense oligonucleotides or hammerhead ribozymes that act against polynucleotides encoding renin [Genbank Accession No. NM_{—}000537], angiotensinogen [Genbank Accession No. NM_{—}000029], angiotensin II receptors (including AT1 [Genbank Accession No. M87290] or AT2), ACE [Genbank Accession No. X16295], aldosterone, aldosterone receptors, and/or any other component of the local renin-angiotensin-aldosterone system.

Antisense polynucleotides include polynucleotides that bind to mRNA and inhibit its translation. Antisense polynucleotides also include polynucleotides that bind to DNA and inhibit its transcription. Hammerhead ribozymes are naturally occurring or synthetic ribonucleic acid oligonucleotides that exhibit enzymatic activity for cleavage of RNAs and that can be engineered to cleave specific RNAs (generally mRNAs) with the same high specificity as protein enzymes. They are used in the same way as antisense oligonucleotides, to target specific mRNAs and knock out a gene product. See, e.g., Tang et al., Cancer Res Oct. 15, 1999; 59(20):5315-22; LaVail et al., Proc Natl Acad Sci USA Oct. 10, 2000; 97(21):11488-93.

For example, use of inhibitors of chymase ([Genbank Accession No. M69136] an enzyme that can convert ANG I to ANGII in one of the “non-ACE” pathways for ANGII formation, and that is found in many organs and cell types including lung and heart), cathepsin D ([Genbank Accession No. NM_{—}001909] an enzyme that perform the same reaction as renin, i.e., conversion of angiotensinogen to ANG I, and that is found in some cell types of the heart, lung and other organs), and cathepsin G ([Genbank Accession No. NM_{—}001911] an enzyme that can convert angiotensinogen directly to ANG II, and that is expressed in some heart and lung cells) including antibodies, antisense mRNA and hammerhead ribozymes, are contemplated according to all of the methods of the invention.

Angiotensin II antagonists are compounds that antagonize the activity of angiotensin II and include antibodies to angiotensin II, angiotensin receptor antagonists and antisense oligonucleotides to angiotensinogen or angiotensin receptor DNA or mRNA, as well as hammerhead ribozymes that degrade such mRNA.

Angiotensin II receptor antagonists are compounds that antagonize angiotensin II receptor, particularly type I (AT1) receptor, and include peptides that bind to the receptor (e.g., as antagonists or competitive antagonists of angiotensin II), such as saralasin, or non-peptide compounds such as losartan or compound L158809, which are selective for receptor subtype AT1, or congeners with AT1 antagonist activity. The compound PD126055 is selective for receptor subtype AT2.

ACE inhibitors include amino acids and derivatives thereof, peptides, including di- and tri-peptides and antibodies to ACE, as well as antisense oligonucleotides to polynucleotides encoding ACE, which intervene in the renin-angiotensin system by inhibiting the activity of ACE, thereby reducing or eliminating the formation of the pressor substance angiotensin II. Classes of compounds known to be useful as ACE inhibitors include carboxylalkyl dipeptide mimics such as enalopril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729) or congeners, carboxyalkyl dipeptide mimics such as cilazapril (U.S. Pat. No. 4,512,924) and benzapril (U.S. Pat. No. 4,410,520), phosphinylalkanoyl prolines such as fosinopril (U.S. Pat. No. 4,337,201) or trandolopril, or acylmercapto and mercaptoalkanoyl prolines such as captopril (U.S. Pat. No. 4,105,776) or zofenopril (U.S. Pat. No. 4,316,906) or congeners.

Aldosterone antagonists include compounds such as spironolactone, canrenone and potassium canrenoate, as well as antisense oligonucleotides to polynucleotides encoding aldosterone or aldosterone receptors.

Caspase inhibitors are inhibitors of any of the cysteine proteases involved in the signaling pathways that promote apoptosis, including Caspase-1, -2, -3, -4, -5, -6, -7, -8, -9, -10, -11 or -12. Examples include ZVAD-cmk or fmk, DEVD-cmk or -fmk or YVAD-cmk or -fmk.

Endonuclease inhibitors include aurintricarboxylic acid (ATA) or zinc salts.

Administration of the therapeutic compound(s) may result in a reduced incidence of pulmonary fibrosis, a later onset of pulmonary fibrosis, slower or arrested progression of the disease or a reduced severity of disease. Thus, therapeutically effective amounts of the therapeutic compound(s) include: amounts effective to inhibit apoptosis of pulmonary epithelial cells, amounts effective to prevent pulmonary fibrosis, including an amount effective to reduce the incidence of pulmonary fibrosis; and amounts effective to slow the progression of pulmonary fibrosis, including an amount effective to delay onset or resolve pulmonary fibrosis, to reduce the severity of pulmonary fibrosis, to prevent or halt further destruction of pulmonary epithelial cells, or to reduce the severity of pulmonary fibrosis. The therapeutic product(s) may not only slow the progression of or reduce the severity of pulmonary fibrosis, but may also arrest or reverse the progression of the disease.

“Pulmonary fibrotic disease” as used herein generally means diseases associated with histological signs or clinical symptoms of pulmonary fibrosis, e.g., increased deposition of collagen, particularly in alveolar septa and peribronchial parenchyma, thickened alveolar septa, decreased gas exchange resulting in elevated circulating carbon dioxide and reduced circulating oxygen levels, decreased lung elasticity which can manifest as restrictive lung functional impairment with decreased lung volumes and compliance on pulmonary function tests, bilateral reticulonodular images on chest X-ray, progressive dyspnea (difficulty breathing), and hypoxemia at rest that worsens with exercise.

Subjects that may be treated according to the methods of the invention include subjects with pulmonary fibrosis, particularly early stage pulmonary fibrosis, and subjects at risk of pulmonary fibrosis. Subjects suffering from pulmonary fibrotic disease include subjects suffering from idiopathic pulmonary fibrosis, sarcoidosis, familial pulmonary fibrosis, pulmonary fibrosis associated with collagen-vascular disorders or vasculitides, histiocytosis X, Goodpasture's syndrome, chronic eosinophilic pneumonia, idiopathic pulmonary hemosiderosis, hypersensitivity pneumonitides; subjects suffering from pulmonary fibrosis caused by inhalation of organic or inorganic dusts, such as coal, crystalline silica and silicates such as asbestos (causing, e.g., silicosis, asbestosis, coal worker's or carbon pneumoconiosis); subjects suffering from pulmonary fibrosis caused by exposure to radiation or toxic agents such as paraquat, caused by an infectious agent, caused by inhalation of noxious gases, aerosols, chemical dusts, fumes or vapors, or drug-induced interstitial lung disease (ILD). Subjects at risk of pulmonary fibrotic disease include subjects undergoing radiation therapy or chemotherapy; subjects with a family history of or genetic factors indicating a predisposition to ILD; subjects in occupations involving exposure to radiation, toxic agents, or inhalation of dusts or noxious vapors, and subjects suffering from infections that may lead to complications that include pulmonary fibrosis.

The therapeutic compound(s) can be administered to subjects at doses ranging from about 1 μg/kg to 100 mg/kg daily, varying in adults and children. The therapeutic compound(s) may be administered as part of a composition including a pharmaceutically acceptable diluent, adjuvant or carrier, and may be administered systemically or topically. Systemic routes include, e.g., oral, intravenous, intramuscular or subcutaneous injection (including into depots for long-term release), or intraocular, retrobulbar, intraventricular, intrathecal (into cerebrospinal fluid), intraperitoneal, intrapulmonary or transdermal routes. The therapeutic compound(s) may be aerosolized or nebulized (in either powder or liquid form) for pulmonary administration or formulated in a spray for nasal administration. Topical routes include administration in the form of salves, ointments, creams, jellies, patches, ophthalmic drops or opthalmic ointments, ear drops, vaginal or rectal suppositories, enemas, or in irrigation fluids (for, e.g., irrigation of wounds).

The therapeutic compound(s) may be administered parenterally via continuous intravenous infusion, via periodic brief intravenous infusions, or by bolus. Smaller doses can be used at shorter intervals, e.g., multiple times daily, or equivalent dosing of the therapeutic product(s) with a longer half-life can be accomplished at longer intervals. The therapeutically effective dose may be adjusted to provide maximum clinical benefit without resulting in excessive toxicity.

The invention also specifically contemplates antagonism of the renin-angiotensin-aldosterone system through negative regulation of gene expression in the lungs through triple helix formation or antisense DNA or RNA, both of which methods are based on the binding of a polynucleotide sequence, usually 20 to 40 bases in length, to a region of DNA involved in transcription or to the mRNA. Triple helix-formation is designed to block RNA transcription from DNA, while antisense RNA hybridization blocks translation of an mRNA molecule into polypeptide. Both techniques have been demonstrated to be effective in model systems. Antisense polynucleotides may be delivered directly to pulmonary cells (including via liposomes or chemical treatments) or through any vector known in the art, particularly viral vectors (e.g., adenovirus, adeno-associated virus, or a retrovirus). See, for example, Phillips et al., Hypertension, 29:177-187, 1997; Phillips et al., Kidney International, 46:1554-1556, 1994. Example 6 shows that antisense oligonucleotides to angiotensinogen were able to block Fas-induced apoptosis.

The dosage of the therapeutic compound(s) may be increased or decreased, and the duration of treatment may be shortened or lengthened as determined by the treating physician. The frequency of dosing will depend on the pharmacokinetic parameters of the agents and the route of administration. The optimal pharmaceutical formulation will be determined by one skilled in the art depending upon the route of administration and desired dosage. See for example, Remington's Pharmaceutical Sciences, 18th Ed. (1990) (Mack Publishing Co., Easton, Pa.) pages 1435-1712, the disclosure of which is hereby incorporated by reference. Such formulations may influence the physical state, stability, rate of in vivo release, and rate of in vivo clearance of the administered therapeutic product(s).

Those of ordinary skill in the art will readily optimize effective dosages and administration regimens as determined by good medical practice and the clinical condition of the individual patient. Regardless of the manner of administration, the specific dose may be calculated according to body weight, body surface area or organ size. Further refinement of the calculations necessary to determine the appropriate dosage for treatment involving each of the above-mentioned formulations is routinely made by those of ordinary skill in the art without undue experimentation, especially in light of the dosage information and assays disclosed herein, as well as the pharmacokinetic data observed in human clinical trials. Appropriate dosages may be ascertained through use of established assays for determining blood levels in conjunction with appropriate dose-response data. The final dosage regimen will be determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. As studies are conducted, further information will emerge regarding the appropriate dosage levels for the treatment of various diseases and conditions.

Co-administration of the therapeutic compound(s) with other agents that treat pulmonary fibrosis or symptoms of pulmonary fibrosis is also contemplated. Such other agents include corticosteroids, cyclophosphamide, chlorambucil, and cyclosporine. See Hunninghake et al., Am. J. Resp. Crit. Care Med. 151:915-918 (1995) and agents discussed therein, the entire disclosure of which is incorporated herein by reference. If the second agent is an ACE inhibitor, an angiotensin II antagonist, an angiotensin II receptor antagonist, a caspase inhibitor, an endonuclease inhibitor, or another compound that modulates the renin-angiotensin system and/or the FAS receptor/ligand system, the dosage of each agent required to exert a therapeutic effect during combinative therapy may be less than the dosage necessary for monotherapeutic effectiveness. Treatment with the therapeutic compound(s) according to the present invention may also provide an added clinical benefit by reducing the severity of complications, e.g., metabolic dysfunction related to compromised gas exchange, associated with pulmonary fibrosis.

Presently preferred therapeutic product(s) include non-thiol ACE inhibitors, angiotensin II antagonists, angiotensin II receptor antagonists, aldosterone antagonists, other renin-angiotensin-aldosterone system inhibitors, FAS receptor/ligand system inhibitors, caspase inhibitors and endonuclease inhibitors.

A protein kinase C inhibitor such as chelerythrine is also another potential therapeutic product for inhibiting pulmonary fibrosis.

“Concurrent administration,” or “co-administration,” as used herein includes administration of the agents, in conjunction or combination, together, or before or after each other. The multiple agent(s) may be administered by the same or by different routes, simultaneously or sequentially, as long as they are given in a manner sufficient to allow all agents to achieve effective concentrations at the site of action.

The screening methods of the invention are amenable to high throughput screening (HTS) assays. HTS permit screening of large numbers (i.e., tens to thousands or more) of compounds in an efficient manner. Cell-based HTS systems are also embraced, including melanophore assays, yeast-based assay systems, and mammalian cell expression systems [Jayawickreme and Kost, Curr. Opin. Biotechnol. 8:629-634 (1997)]. Automated and miniaturized HTS assays are particularly preferred [Houston and Banks, Curr. Opin. Biotechnol. 8:734-740 (1997)]. HTS assays are designed to identify “hits” or “lead compounds” having the desired inhibitory property, from which modifications can be designed to improve the desired property. Chemical modification of the “hit” or “lead compound” is often based on an identifiable structure/activity relationship between the “hit” and one or more of the binding partner polypeptides.

There are a number of different libraries used for the identification of specific small molecule inhibitors, including, (1) chemical libraries, (2) natural product libraries, and (3) combinatorial libraries comprised of random peptides, oligonucleotides or organic molecules.

Chemical libraries consist of structural analogs of known compounds or compounds that are identified as “hits” or “leads” via natural product screening. Natural product libraries are derived from collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998), incorporated by reference herein. Combinatorial libraries are composed of large numbers of peptides, oligonucleotides or organic compounds as a mixture. They are relatively easy to prepare by traditional automated synthesis methods, PCR, cloning or proprietary synthetic methods. Of particular interest are peptide and oligonucleotide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997), incorporated by reference herein.

Other aspects and advantages of the present invention will be understood upon consideration of the following illustrative examples. Example 1 addresses activity of captopril on FAS-induced apoptosis in a pulmonary epithelial cell line. Example 2 addresses activity of a non-thiol ACE inhibitor and an angiotensin II receptor antagonist on FAS-induced apoptosis in a pulmonary epithelial cell line. Example 3 addresses induction of AEC apoptosis by angiotensin II or angiotensinogen and the effects of ACE inhibitors and angiotensin II receptor antagonists. Example 4 addresses bleomycin-induced epithelial cell apoptosis and lung fibrosis and the effects of captopril and caspase inhibitors. Example 5 addresses the identification of fibroblast-derived inducers of AEC apoptosis as angiotensin peptides. Example 6 addresses the requirement for expression and activity of angiotensin II and its receptor in Fas-induced apoptosis by AEC. Example 7 addresses the inhibition of amiodarone-induced toxicity by administration of ACE inhibitors and AT1 selective antagonists.

### EXAMPLE 1

Effect of Captopril on FAS-Induced Apoptosis

Experiments were conducted to study the effect of captopril on FAS-induced apoptosis in a human lung epithelial cell line.

Reagents and Materials. Monoclonal activating antibodies to human FAS (clone CH-11, purified mouse IgM) were obtained from Upstate Biotechnology, Saranac Lake, N.Y. Purified non-immune mouse IgM was obtained from Sigma Chemical. Saint Louis, Mo. Monoclonal non-activating antibodies to human FAS (clones 5F-7 and 5F-9) were obtained from Kamiya Biomedical Company, Seattle Wash. All other materials were from sources described in Uhal et al., Am. J. Physiol. 269:L819-L828 (1995), or were of reagent grade.

Cell Culture. The human lung adenocarcinoma cell line A549 was obtained from American Type Culture Collection and cultured in Ham's F12 medium supplemented with 10% fetal bovine serum (FBS). Cells were seeded on 12 mm sterile glass cover slips in 24-well chambers at a density of 20,000 cells per well. All experiments were conducted at subconfluent densities of 80-90% in Ham's F12 medium supplemented with 1% fetal bovine serum. Antibodies and captopril were diluted with Ham's F12 medium and were applied for 20 h at 37° C. in a 5% CO₂ incubator.

Fluorescence Detection of Apoptosis. Detection of apoptotic cells with propidium iodide (PI) was conducted as described in Uhal et al., Am. J. Physiol. 269:L819-L828 (1995) following digestion of ethanol-fixed cells with DNAse-free RNAse in PBS containing 5 ug/ml PI. In these assays detached cells were retained by centrifugation of the 24-well culture vessels during fixation with 70% ethanol. In situ end-labeling of fragmented DNA (ISEL) was conducted as described in Gorczyka et al., Canc. Res. 53:1945-1951 (1993). Briefly, ethanol was removed by rinsing cover slips in distilled water for at least 10 minutes. The cover slips were then placed in saline sodium citrate solution (SSC) (0.3 M NaCl and 30 mM sodium citrate in water, pH 7.0) at 37° C. for 20 minutes. After four rinses in PBS and four rinses in Buffer A (50 mM Tris/HCl, 5 mM MgCl, 10 mM β-mercaptoethanol and 0.005% BSA in water, pH 7.5), the cover slips were incubated at 18° C. for two hours with ISEL solution (0.001 mM biotinylated deoxyuridine trisphosphate (Bio-dUTP), 20 U/ml DNA Polymerase 1 and 0.01 mM each of dATP, dCTP and dGTP in Buffer A). The sections were rinsed thoroughly five times with Buffer A and three additional times in 0.5 M PBS. Incorporated bio-UTP was detected by incubation for one hour at 37° C. with avidin-rhodamine; the cover slips were then rinsed in distilled water three times and mounted under Fluoromount Solution (Southern Biotechnology Associates, Birmingham, Ala.).

Caspase Assays. The enzymatic activities of Caspase 1/interleukin-1b-converting enzyme (ICE) and Caspase 3/CPP32/YAMA were determined in intact A549 cell cultures pre-incubated under conditions identical to those used for morphologic assays of apoptosis. After a 20 h incubation with antibodies and/or captopril, cells were trypsinized from the culture vessels and resuspended in serum-free culture medium containing anti-FAS antibodies and/or captopril at the same concentrations as during the preincubation. After one hour, fluorogenic peptide substrates specific to each enzyme were added separately to cuvettes containing cell suspension for measurement of total activity in intact cells. For measurement of Caspase 1/ICE activity, the peptide substrate N-Acetyl-Tyr-Val-Ala-Asp-7-amino-4-methylcoumarin (Ac-YVAD-AMC, Pharmingen, San Diego, Calif.) was used at 50 uM final concentration. For Caspase 3/CPP32/YAMA, the peptide substrate N-Acetyl-Asp-Glu-Val-Asp-7-amino-4-methylcoumarin (Ac-DEVD-AMC, Upstate Biotechnology, Saranac Lake, N.Y.) was added at 200 uM final concentration. Production of fluorescent product over time was monitored in a spectrofluorometer at 380 nm excitation and 450 nm emission. Both enzyme assays were linear with time and protein concentration.

Monoclonal antibodies which activate the FAS receptor induced epithelial cell apoptosis that was detectable by chromatin condensation, nuclear fragmentation, DNA fragmentation and increased activities of Caspases 1 and 3. Epithelial cell apoptosis was not induced by isotype-matched non-immune mouse immunoglobulins nor by non-activating anti-FAS monoclonal antibodies. When applied simultaneously with anti-FAS antibodies, 50 ng/ml captopril completely abrogated apoptotic indexes based on morphology, DNA fragmentation and inducible Caspase I activity, and significantly decreased the inducible activity of Caspase 3. Inhibition of apoptosis by captopril was concentration-dependent with an IC₅₀ of 70 pg/ml. These data indicate that the inhibitory actions of captopril on pulmonary fibrosis may be related to prevention of lung epithelial cell apoptosis.

Spontaneous or stimulated apoptosis was quantitated as described in Uhal et al., Am. J. Physiol. 269:L819-L828 (1995), in the human lung epithelial cell line A549 by fluorescence detection of chromatin condensation and nuclear fragmentation. The human lung epithelial cell line A549 was cultured on glass cover slips as described in Uhal, et al., Am. J. Physiol. 269:L819-L828 (1995). At the end of the test period, detached cells were retained by centrifuging the culture vessel during fixation of cells in 70% ethanol. Fixed cells were incubated for 30 minutes with 5 ug/ml propidium iodide (PI) in PBS containing DNAse-free RNAse; under these conditions, red fluorescence (>570 nm) is specific for DNA. As in Uhal et al., Am. J. Physiol. 269:L819-L828 (1995), apoptotic cells were identified by the presence of discrete nuclear fragments containing condensed chromatin. Human lung A549 cells were cultured for 20 h in the presence of equivalent concentrations (500 ng/ml) of monoclonal antibody CH-11 (FAS mAb), purified IgM fraction of nonimmune mouse immunoglobulins (IgM), monoclonal antibody 5F-7 (mAb2), and monoclonal antibody 5F-9 (mAb3). Apoptotic cells were scored by the nuclear morphology assay discussed above. A significant difference (p<0.01 compared to control (no additives)), was observed between the means±S.E.M. (at least 4 determinations per condition) of the cells exposed to FAS mAb as compared to the control or other antibody treatments as determined by ANOVA and Dunnett's test. This assay indicated that apoptosis was induced in A549 cells by the monoclonal activating antibody CH-11 but not by isotype-matched non-immune mouse IgM at equivalent concentrations. The monoclonal non-activating antibodies 5F-7 and 5F-9 also failed to induce apoptosis in A549 cells; the latter results are consistent with recent findings described in Wen et al., Amer. J. Physiol. 273:L921-L929 (1997), using the same antibody preparations, which did not activate FAS in the absence of other inducers.

The more sensitive assay of in situ end-labeling (ISEL) of fragmented DNA revealed a potent inhibitory action of captopril on FAS-induced cell death in A549 cells. Ethanol-fixed A549 cells on glass cover slips were subjected to ISEL labeling of fragmented DNA. DNA fragments labeled with biotinylated UTP were detected with rhodamine-conjugated avidin. A549 cells were exposed to either 500 ng/ml anti-FAS antibody (CH-11) under the same conditions as described above or in the presence of 50 ng/ml captopril. Although captopril alone had no significant effect on basal ISEL labeling, 50 ng/ml captopril essentially abolished the generation of ISEL-positive cells in response to ligation of FAS antigen by anti-FAS antibodies (significant differences between the means±S.E.M. of at least 4 determinations, p<0.001 compared to control by ANOVA and Dunnett's test). ISEL-positive A549 cells were quantitated in at least four separate culture vessels exposed to the anti-FAS antibody CH-11 at 500 ng/ml and/or 50 ng/ml captopril.

The same concentration of captopril also abrogated FAS-induced stimulation of Caspase 1 (ICE) (p<0.01 compared to control) and Caspase 3/CPP32/YAMA (p<0.01 compared to FASmAb treatment alone) activities. A549 cells were exposed as described above to anti-FAS monoclonal antibody CH-11 (FASmAb) alone or in the presence of 50 ng/ml captopril. Cells were then analyzed in suspension culture for the activity of Caspase 1/ICE with peptide substrate Ac-YVAD-AMC or for activity of Caspase 3/CPP32/YAMA with peptide substrate Ac-DEVD-AMC as described above. Both Caspase 1/ICE and Caspase 3/CPP32/YAMA are cysteine proteases suspected to be involved in the mechanism of apoptosis. See Mundle et al., Pathobiology 64:161-170 (1996).

The effect of captopril on apoptosis was found to be concentration-dependent. A549 cells were exposed to anti-FAS antibody CH-11 as described above, in the presence of 0.005 ng/ml (5 pg/ml) to 50 ng/ml captopril. Inhibition of apoptosis was detected as the decrease in ISEL-positive cells, scored as described above. Inhibition of FAS-induced ISEL labeling was measurable at a concentration of 5 pg/ml captopril and exhibited an IC₅₀ of 70 pg/ml. Inhibition of apoptosis was maximal (approximately 85-90%) at 50 ng/ml captopril, a concentration known to be physiologically attainable in humans and to be maximally inhibitory for ACE. See Duchin et al., Clin. Pharmacokinet. 14:241-259 (1988).

This data suggests an entirely different mechanism of antifibrotic action by captopril. Inhibition of apoptosis by an ACE inhibitor has previously been demonstrated only in cell types of hematopoietic lineage. Deas et al. Int. Immunol. 9:117-125 (1997) demonstrated inhibition of FAS-induced apoptosis in activated human peripheral T cells by captopril and other thiol compounds, but not by non-thiol antioxidants. Those results led the authors to speculate that the inhibition of T-cell apoptosis was the result of sulfhydryl redox regulation of critical molecules involved in the apoptotic signaling cascade.

The caspases are cysteine proteases critical to the signaling of apoptosis and are sensitive to sulfhydryl redox status. See Mundle et al., Pathobiology 64:161-170 (1996). The data from these experiments showed that at least two of these enzyme activities, Caspase 1/ICE and Caspase 3/CPP32/YAMA, are inhibited in situ by exposure of the intact cell to captopril. These results suggest that captopril and other thiol compounds may inhibit lung epithelial cell apoptosis through direct inhibition of Caspase 1 and 3 activities and/or the activities of other cysteine proteases required for the induction of apoptosis.

### EXAMPLE 2

Effect of Lisinopril and Saralasin on FAS-Induced Apoptosis

Experiments were conducted to investigate the effect of a non-thiol ACE inhibitor and an angiotensin II receptor antagonist on apoptosis in a pulmonary epithelial cell line induced by FAS activation. Methods and materials were as described in Example 1 above.

Apoptosis was induced in the A549 human AEC-derived cell line and in primary rat AEC (isolated type II pneumocytes) by incubation with receptor-activating antibodies to FAS (clone CH-11, 200 ng/ml) or by recombinant human FasLigand (FASL) (25 ng/ml). Apoptosis of both cell types was inhibited 61% and 87%, respectively, by the non-thiol ACE inhibitor lisinopril (500 ng/ml, both p<0.01). Apoptosis also was inhibited by 71% and 92%, respectively, by the angiotensin II (ATII) receptor antagonist saralasin (5 mg/ml, both p<0.01). By Western and Northern analyses, exposure to FAS antibodies or FASL stimulated the expression of both angiotensinogen and ATII receptors, and significantly increased the amount of free ATII present in the culture medium of FAS-activated cells. Enzyme assay revealed ACE-like and renin-like activities present in whole cell lysates of both cell types. Purified ATII (100 uM) in serum-free medium induced apoptosis in either cell type (both p<0.01), and antibodies to ATII blocked either ATII- or FAS-induced apoptosis. These results indicate that activation of FAS in AEC stimulates expression of the renin-angiotensin system, and that this system is required for the execution of apoptosis by FAS. They also suggest a mechanism for the attenuation of experimental lung fibrogenesis by ACE inhibitors and related compounds.

### EXAMPLE 3

Induction of Apoptosis by Angiotensin II and its Receptor; Effect of ACE Inhibitors and Angiotensin II Receptor Antagonists

The results presented in EXAMPLES 1 and 2 above demonstrated potent inhibition of apoptosis in the human AEC-derived A549 cell line by the ACE inhibitor captopril. The inhibition of apoptosis exhibited a concentration-dependence similar to that for captopril inhibition of angiotensin converting enzyme in other cell types. See Muns et al., J. Cell. Biochem. 53:352-359 (1993). Further experiments were conducted to determine whether AEC apoptosis might be induced by angiotensin II (“ANGII”) or angiotensinogen through pathways involving ACE and the angiotensin II receptor (“ANGIIR”).

Reagents and Materials. Purified angiotensin II (ANGII), angiotensinogen, lisinopril, saralasin and antibodies to ANGII and angiotensinogen, respectively, were obtained from Sigma Chemical, Saint Louis, Mo. Fluorescein-conjugated Annexin V was obtained from Pharmingen (San Diego, Calif.). PCR primers were synthesized by GeneMed Synthesis, San Francisco, Calif. All other materials were from sources described in Uhal, et al., Am. J. Physiol. 275:L1013-1017 (1998) and Uhal et al., Am. J. Physiol. 275:L998-L1005 (1998), or were of reagent grade.

Cell Culture. The human lung adenocarcinoma cell line A549 was obtained from American Type Culture Collection and cultured in Ham's F12 medium supplemented with 10% fetal bovine serum (FBS). Primary alveolar epithelial cells were isolated from adult male Wistar rats as described in Uhal et al., Am. J. Physiol. Suppl. (Oct.) 261:110-117 (1991) and Uhal et al., Am. J. Physiol. 269:L819-L828 (1995). The primary cells were studied at day two of culture, a time at which they are type II cell-like by accepted morphologic and biochemical criteria, see Uhal, Am. J. Physiol. 272:L1031-L1045 (1997). All preparations were of greater than 90% purity as assessed by acridine orange staining, as discussed in Uhal, et al., Am. J. Physiol. Suppl. (October) 261:110-117 (1991). All cells were seeded in 24-well or 6-well chambers, and all experiments were conducted at subconfluent densities of 80-90% in serum-free Ham's F12 medium. Test reagents were diluted with Ham's F12 medium and were applied for 20 h at 37° C. in a 5% CO₂ incubator.

Detection of Apoptosis. Detection of apoptotic cells with propidium iodide (PI) was conducted as described in Uhal, et al., Am. J. Physiol. 275:L1013-1017 (1998) following digestion of ethanol-fixed cells with DNAse-free RNAse in PBS containing 5 ug/ml PI. Assay of Annexin V binding by flow cytometry was performed essentially as described for human lung fibroblasts in Uhal, et al., Am. J. Physiol. 275:L998-L1005 (1998) after trypsinization of cells from culture vessels. Assay of Caspase-3 activity was performed on viable suspension cultures as reported in Uhal, et al., Am. J. Physiol. 275:L1013-1017 (1998). In all assays, detached cells were retained by centrifugation of the culture vessels during fixation with 70% ethanol, or by retention of culture media and recovery by centrifugation before assay.

For microscopy, cells were cultured on glass cover slips, incubated with test reagents and fixed with 70% ethanol. The fixed cells were immunolabeled overnight at 4° C. with monoclonal antibodies to cytokeratins 7 and 19 (Chemicon International, Temecula, Calif.), and then for one hour at 37° C. with anti-mouse IgG-rhodamine and DNAse-free RNAse (Boehringer Mannheim, Indianapolis, Ind.) The cover slips were then rinsed in distilled water three times and mounted under Fluoromount Solution (Southern Biotechnology Associates, Birmingham, Ala.) containing 5 ug/ml propidium iodide. Photomicroscopy was performed as described in Uhal, et al., Am. J. Physiol. 275:L1013-1017 (1998). Cells exhibiting nuclear fragmentation and chromatin condensation were scored in four randomly selected fields per culture well in at least three culture vessels per experimental condition, and were expressed as a percentage of the total cells within the same field (minimum of 100 cells/field).

RT-PCR. Total RNA was isolated by the RNeasy Mini protocol (Qiagen Co., Santa Clarita, Calif.). To synthesize cDNA by RT-PCR, 3 ug of purified RNA was reverse-transcribed using 2 uM oligo-dT, 50 mM Tris-HCl (pH 8.3), 75 mM KCl, 3 mM MgCl₂, 0.01 mM DTT, 0.2 mM of each dNTP, 1 U/ul RNase inhibitor (RNasin), and 2 U of avian myoblastosis virus reverse transcriptase (AMV-RT) (Promega Co., Madison, Wis.) in a total volume of 30 ul. The reaction was performed for 1 hour at 45° C.

PCR amplification was performed with 10 ul aliquots of cDNA, obtained as described above, equivalent to 1 ug of the starting RNA. The reaction was performed in 50 ul PCR buffer containing 5 mM MgCl₂, 5 ug/ml of each 5′ and 3′ primer (see below), 2 ul of 10 mM dNTPs and 1 U of Taq polymerase (Promega Co, Madison, Wis.) with a Perkin Elmer PCR amplifier. The PCR reactions included 20 cycles of denaturation at 95° C. for 30 sec, annealing of primers at 45° C. for 30 sec, and elongation of the chain at 72° C. for 1 min using Taq DNA polymerase. Samples were stored at 4° C. Negative controls lacked DNA. The identity of expressed genes was determined by concordance with the expected size of the PCR product after electrophoresis in 1.6% agarose gels.

For RT-PCR from the human A549 cell line, the following primers were used: a “coding strand” oligonucleotide having the sequence 5′ ACTGGCTGACTTATGCTTTTTACT 3′ [SEQ I.D. NO. ______] and a “noncoding strand” oligonucleotide having the sequence 5′ AGAAAAGGAAACAGGAAACCCAGTA 3′ [SEQ I.D. NO. ______] were used for amplification of angiotensin II receptor type 1 to generate a PCR product of 414 bp. See Arnal et al., Am. J. Physiol. 267:H1777-H1784 (1994).

A “coding strand” oligonucleotide having the sequence 5′ CCTTTTGGCTACTCTTCCTCTATGG 3′ [SEQ I.D. NO. ______] and a “noncoding strand” oligonucleotide having the sequence 5′ TTGGTCACGGGTTATCCTGTTCTTC 3′ were used for amplification of angiotensin II receptor type 2 to generate a product of 414 bp. See id.

A “coding strand” oligonucleotide having the sequence 5′ AGTACAACAAGATCCTGTTG 3′ [SEQ I.D. NO. ______] and a “noncoding strand” oligonucleotide having the sequence 5′ GATGTGGCCATCACATTCGTCAGA 3′ were used for amplification of angiotensin converting enzyme to generate a PCR product of 499 bp. Id.

For RT-PCR of rat-specific gene products, the following primers were used: A “coding strand” oligonucleotide having the sequence 5′ GAGAGGATTCGTGGCTTGAG 3′ [SEQ I.D. NO. ______] and a “noncoding strand” oligonucleotide having the sequence 5′ GAGACACGTGAGAAGGAACA 3′ [SEQ I.D. NO. ______] were used to amplify angiotensin II receptor type 1 to generate a PCR product of 235 bp. See Du et al., Hypertension 25:872-877 (1995).

A “coding strand” oligonucleotide having the sequence 5′ ATGAAGGACAACTTCAGTTTTGC 3′ [SEQ I.D. NO. ______], and a “noncoding strand” oligonucleotide having the sequence 5′ CAAGGGGAACTACATAAGATGGC 3′ [SEQ I.D. NO. ______], were used to amplify angiotensin II receptor type 2 to generate a product of 499 bp. See Shanmugam et al., Kidney International 47:1095-1100 (1995).

A “coding strand” oligonucleotide having the sequence 5′ GTCAGCTTCATCATCCAGTT 3′ [SEQ I.D. NO. ______] and a “noncoding strand” oligonucleotide having the sequence 5′ AGGAAGAGCAGCAGCCACTG 3′ were used to amplify angiotensin converting enzyme to generate a PCR product of 304 bp. See Katwa et al., Cardiovascular Res. 29:57-64 (1995).

Statistics. All values reported are the mean±S.E.M. Significant differences between treatment means were determined by ANOVA followed by the indicated post-hoc analysis. All experiments were performed at least two times and the data were combined.

Purified ANGII induced dose-dependent apoptosis in both the human AEC-derived A549 cell line and in primary type II pneumocytes isolated from adult Wistar rats. Apoptosis in these cells was detected by nuclear and chromatin morphology, Caspase-3 activity and increased binding of Annexin-V. Apoptosis was also induced in primary rat AEC by purified angiotensinogen. The nonselective angiotensin II receptor (ANGIIR) antagonist saralasin completely abrogated both ANGII- and angiotensinogen-induced apoptosis at a concentration of 50 ug/ml. Analysis of mRNA levels by reverse transcription-polymerase chain reaction (RT-PCR) assay indicated that each cell type expressed the ANGIIR subtypes 1 and 2 and angiotensin converting enzyme (ACE). The non-thiol ACE inhibitor lisinopril blocked apoptosis induced by angiotensinogen, but not apoptosis induced by purified ANGII. These results indicate that both primary rat alveolar epithelial cells and the A549 human AEC-derived cell line express renin-like and ACE-like activities as well as functional ANGII receptors capable of inducing apoptosis. These data demonstrate the presence of a functional ANGII-dependent pathway of apoptosis in human and rat AEC, and suggest a role for ANGII receptors and angiotensin converting enzyme in the induction of AEC apoptosis in vivo.

Apoptosis in alveolar epithelial cells was quantitated by fluorescence detection of chromatin condensation and nuclear fragmentation in alcohol-fixed cells stained with propidium iodide. See Uhal, et al., Am. J. Physiol. 275:L1013-1017 (1998). Primary alveolar epithelial cells (AEC) were incubated with purified angiotensin II (ANGII, 5.0 uM) in serum-free medium or medium supplemented with 1% fetal bovine serum (FBS). After 20 h, the culture vessel was centrifuged to retain detached cells and labeled with anticytokeratin antibodies and propidium iodide. Fluorescence due to chromatin-bound propidium iodide was observed in the absence of anti-cytokeratin. Condensed chromatin was observed in fragmented nuclei within cells labeled with propidium iodide and containing heavily decorated cytokeratin filaments. Double labeling with PI and monoclonal antibodies to cytokeratins 7 and 19 identified cells with both apoptotic nuclear morphology and positive anticytokeratin immunoreactivity. Although apoptotic cells with negative immunoreactivity and the spindle-shaped morphology of fibroblasts were occasionally observed within the primary rat lung cell preparations, these were not scored.

Purified angiotensin II (ANGII) induced apoptosis in a concentration-dependent manner in both the human A549 cell line and in primary rat AEC. In A549 cells, the percent of apoptotic induction was significantly elevated at concentrations of ANGII as low as 0.01 uM (p<0.05 for 0.01 and 0.1 uM ANGII versus corresponding control and versus maximum stimulatory dose, and p<0.05 for 1.0, 10 and 100 uM ANGII versus corresponding control, but not significantly different from maximum dose, both by ANOVA and Student-Newman-Keul's test). In rat AEC, the percent of apoptotic induction was significantly elevated at concentrations of ANGII as low as 0.005 uM (p<0.05 for 0.005, 0.01, and 0.1 uM ANGII versus corresponding control and versus maximum stimulatory dose, and p<0.05 for 1.0, 10 and 100 uM ANGII versus corresponding control, but not significantly different from maximum dose, by ANOVA and Student-Newman-Keul's test). The absolute values for basal, i.e., 0.0 dose, apoptosis were: A549 serum-free, 2.29±0.33%; A549+FBS (i.e., media supplemented with 1% fetal bovine serum): 2.02±0.25%; AEC serum-free: 1.87±0.41%; and AEC+FBS: 1.97±0.37%. The observed EC₅₀ for apoptosis by ANGII was 50 nM for A549 cells and 10 nM for primary rat AEC; the presence or absence of medium growth factors (i.e., ±FBS) had no influence on maximal induction of apoptosis by ANGII.

To confirm the induction of apoptosis, Annexin V binding and the activity of Caspase 3/CPP32[YAMA were assessed in both human and rat AEC exposed to purified ANGII peptide. Primary AEC or A549 cells were incubated with purified angiotensin II (5.0 uM) as described above, and were trypsinized from the culture vessels for assay of Annexin-V binding, see Uhal et al., Am. J. Physiol. 275:L998-L1005 (1998), or Caspase 3 activity assay. See Uhal et al., Am. J. Physiol. 275:L,1013-1017 (1998).

Annexin V-positive cells comprised 9.6% and 10.4% (control) and 38.2% and 43.6% (+ANGII) of the total cell population in two separate experiments. Thus, incubation with 5.0 uM ANGII increased the percentage of Annexin V-positive primary rat AEC from 10.0% to 40.9%. Incubation with 5.0 uM ANGII also significantly increased the activity of Caspase-3 in intact AEC of either human or rat origin (p<0.05 compared to control by ANOVA and Student-Newman-Keul's test).

Apoptosis also was induced in primary alveolar epithelial cells by purified angiotensinogen. Primary rat AEC were incubated with purified angiotensin II (ANGII, 5.0 uM) or angiotensinogen (ANG, 5.0 uM) in the presence or absence of lisinopril (500 ng/ml) or saralasin (50 ug/ml). Absolute value for basal (no additive) apoptosis was 1.83±0.21%. At the maximal concentration of 5 uM, ANG induced apoptosis with the same potency as did ANGII (both p<0.01 versus basal).

The nonselective ANGII receptor antagonist saralasin completely inhibited AEC apoptosis induced by either ANGII or purified angiotensinogen (significantly different from ANGII- or ANG-stimulated (both p<0.001) but not significantly different from basal), suggesting a mechanism mediated through ANGII interaction with one or both of its receptors.

The non-thiol angiotensin converting enzyme inhibitor lisinopril also blocked apoptosis in response to angiotensinogen (significantly lower than ANGII- or ANG-stimulated, both p<0.001, but not significantly different from basal), but did not block apoptosis stimulated by purified ANGII (significantly higher than basal (p<0.001) but not significantly different from ANGII-stimulated) (all comparisons were by ANOVA and Student-Newman-Keuls test). Purified angiotensinogen was not capable of inducing apoptosis in the human A549 cell line, suggesting that these cells may not express ACE-like or renin-like activities sufficient to convert angiotensinogen to ANGII. However, apoptosis induced by purified ANGII in the A549 cell line was completely inhibited by saralasin at the same concentration (50 ug/ml) which abrogated both ANGII- and ANG-induced apoptosis of the primary AEC.

To determine if alveolar epithelial cells express ANGII receptors and angiotensin converting enzyme in vitro, RT-PCR was performed on A549 and AEC-derived total RNA with human- and rat-specific primers for the type 1 and type 2 ANGII receptors and ACE. Total RNA was isolated from the human A549 cell line or from primary rat AEC. RT-PCR amplification was performed with primers specific for the human or rat ANGII receptors type I (RT1) and type II (RT2) or angiotensin converting enzyme (ACE) (see details above). The results indicate that both A549 cells and primary rat AEC express mRNAs for both the type 1 and type 2 ANGII receptors and ACE. All major PCR products were of the correct length predicted.

Apoptosis by cells of epithelial origin has not previously been demonstrated in response to angiotensin peptides. The effective concentration of ANGII on primary AEC (EC₅₀=10 nM) is similar to that reported by Dimmeler et al. for ANGII-induced apoptosis of human umbilical vein endothelial cells (HUVECs), id., but lower than that observed here for the human A549 cell line. Further, ANGII elicited a 4- to 5-fold activation of apoptosis in AEC, which is of similar magnitude to that of ANGH-induced apoptosis of cardiac myocytes, see Kajstura et al., J. Mol. Cell. Cardiol. 29:859-870 (1997), but induced only a 2- to 3-fold activation in the HUVEC cell line. Whether these disparities reflect cell type or species differences, or the poorly differentiated and transformed phenotypes of the A549 and HUVEC cell lines, awaits further study.

As discussed in Uhal et al., Am. J. Physiol. 269:L819-L828 (1995), the scoring of cells on the basis of nuclear fragmentation provides a much less ambiguous assay than does the now-popular TUNEL assay, which does not discriminate between apoptotic, necrotic or autolytic cells. See Grasl-Kraup et al., Hepatology 21:1465-1468 (1995). However, because it relies on the appearance of morphologic changes that occur late in apoptosis, the nuclear morphology assay underestimates the true fraction of apoptotic cells. The fact that the apoptotic indexes determined by Annexin V binding (10.0% basal and 40.9% ANGII-stimulated) were significantly greater than those scored by the propidium iodide assay (1.87±0.41% at 0.0 dose versus 9.46±0.89% at 100 uM ANGII) is consistent with the known ability of Annexin V to identify cells in both early and advanced stages of apoptosis. See Uhal et al., Am. J. Physiol. 269:L8]9-L828 (1995). Even so, the fact that the observed increase in Caspase 3 activity in response to ANGII (50%) was lower than that observed above for FAS-induced apoptosis in the same cell type (4-fold, see Uhal, et al., Am. J. Physiol. 275:L1013-1017 (1998)) suggests that the maximum fraction of epithelial cells induced to undergo apoptosis in response to ANGII likely did not exceed 50%. Regardless, the ability of purified ANGII to induce significant increases both in the binding of Annexin V and in the activity of Caspase 3 supports the reliability of the propidium iodide nuclear morphology assay as a specific measure of changes in the apoptotic index.

The lowest concentration of purified ANGII capable of inducing a statistically significant increase in apoptosis of primary AEC (5 nM) was only slightly higher than the mean concentration of ANGII in arterial plasma (85 pg/ml, or 0.1 nM) obtained from patients with adult respiratory distress syndrome. See Wenz et al., Chest 112:478-483 (1997). Given the relatively short half-life (0.6 minutes) for ANGII in plasma, see Vemace et al., Hypertension 23:853-856 (1994), it seems reasonable that the local concentration of ANGII in the vicinity of cells which synthesize angiotensinogen and converting enzymes might easily exceed the threshhold required for the induction of apoptosis in the alveolar epithelium.

Blockade of ANGII-induced apoptosis by the nonselective ANGII receptor antagonist saralasin supports the conclusion that the induction of apoptosis occurs through at least one of the ANGII receptors. RT-PCR demonstrated the presence of both the type 1 (AT1) and type 2 (AT2) receptors, which appear to be constitutively expressed in A549 and primary epithelial cells at similar levels. Further experiments showed that the AT1-selective antagonist L158809 could block ANGII-induced or (see Example 6 below) Fas-induced apoptosis of either A549 cells or primary rat AEC, but the AT2-selective antagonist PD126055 was unable to significantly inhibit apoptosis by either inducer. In light of the findings that both cell types express the mRNAs for both the type 1 and type 2 ANGII receptors, these data suggest that the type 1 ANGII receptor is primarily the subtype that mediates AEC apoptosis in response to either purified ANGII or Fas (see Example 6 below), and that other type 1-selective receptor antagonists will have the same action on apoptosis of AEC.

The ability of purified angiotensinogen to induce apoptosis in primary AEC suggests that these cells express renin-like and ACE-like activities capable of converting ANG to ANGII. That this conversion is required for the induction of apoptosis by ANG is supported by the ability of lisinopril to abrogate apoptosis induced by ANG, but not that induced by ANGII. Although RT-PCR assays suggest that both A549 cells and primary AEC express ACE, it is possible that both cell types express other peptidases, such as chymase or cathepsins, which also cleave ANGI and angiotensinogen, respectively. See Akasu et al., Hypertension 32:514-520 (1998).

On the other hand, the inability of angiotensinogen to induce apoptosis of A549 cells, in contrast to its potent induction in the primary cell isolates, suggests that A549 cells, at least under basal culture conditions, may not express sufficient renin-like activities to convert the angiotensinogen required to elicit an ANGII-dependent response. The fact that the ACE inhibitor captopril abrogated A549 cell apoptosis induced by anti-FAS antibodies, see EXAMPLE 1 above; see also Uhal, et al., Am. J. Physiol. 275:L1013-1017 (1998), suggests that renin or renin-like activities in these cells may be induced by the activation of FAS.

### EXAMPLE 4

Abrogation of Bleomycin-Induced Epithelial Apoptosis and Lung Fibrosis by Captopril and by a Caspase Inhibitor

Experimental lung fibrosis induced by bleomycin is a well-studied model of fibrogenesis. See, e.g., Hagimoto, et al., Am. J. Respir. Cell Mol. Biol. 16:91-101 (1997); Thrall, R. et al., Am. J. Pathol. 95:117-130 (1979); and Young et al., Environ. Health Perspect. 101:56-61(1993). Although the ability of bleomycin to induce apoptosis in alveolar macrophages has been documented, little was previously known about the direct effect of bleomycin on apoptosis or its regulation in lung epithelial cells.

Experiments were conducted to determine the ability of the ACE inhibitor captopril or the ICE-family caspase inhibitor ZVAD-fmk (which has no capacity to inhibit ACE) to inhibit experimental lung fibrogenesis in response to bleomycin and to inhibit apoptosis of lung epithelial cells.

Adult male Wistar rats were given intratracheal instillations of 8 U/kg bleomycin sulfate (BLEO) or sterile saline. Two days before the start of BLEO administration, two subgroups of BLEO rats received captopril in the drinking water or daily intraperitoneal injections of ZVAD-fmk. Control animals received BLEO and ZVAD-fmk vehicles alone. Lung fibrosis was assessed by picrosirius red (PR) quantitation of lung collagen and by hydroxyproline (HP) assay at 14 days after BLEO administration. At the same time point, fragmented DNA in lung epithelial cells was detected by in situ end labeling (ISEL). By PR staining, BLEO significantly increased both alveolar septal and peribronchial collagen by 100% and 133%, respectively (p<0.01). BLEO also increased ISEL in AEC and airway epithelium. In vivo administration of captopril or ZVAD-fmk inhibited the accumulation of lung collagens by 91% and 85%, respectively (p<0.01); assay of total lung HP confirmed the results of the PR assay. Both agents also inhibited ISEL labeling in AEC by 99 and 81%, respectively, and in airway epithelial cells by 67 and 63%, respectively. These data show that the ability of captopril to inhibit experimental lung fibrogenesis is related to its ability to abrogate lung cell apoptosis. They also demonstrate the potential of caspase inhibitors for the prevention of lung fibrogenesis.

Reagents and Materials. Bleomycin sulfate and captopril were obtained from Sigma Chemical Corp., Saint Louis, Mo. The peptide inhibitor of the ICE-family caspase, ZVAD-fmk (N-benzylcarboxy-Val-Ala-Asp-[O-Me]-CH₂F), was obtained from Kamiya Biomedical, Seattle, Wash. The peptide caspase inhibitors DEVD-fmk (Asp-Glu-Val-Asp-[O-Me]-CH2F) and YVAD-cmk (Tyr-Val-Ala-Asp-[O-Me]-CH₂Cl) were obtained from Pharmingen, San Diego, Calif. Alkaline phosphatase-conjugated streptavidin, digoxigenin-labeled deoxyuridine triphosphate (dig-dUTP) and biotinylated deoxyuridine triphosphate (bio-dUTP) were obtained from Boerhinger Mannheim, Indianapolis, Ind. Reagents for detection of alkaline phosphatase and other secondary reagents for in situ end-labeling of DNA were obtained from sources described in Uhal, et al., Am. J. Physiol. 275:L1192-L1199 (1998).

Animals and Induction of Pulmonary Fibrosis. Forty adult male Wistar rats, 175-200 grams, were divided into four groups; three groups received a single intratracheal instillation of 8 units per kilogram of bleomycin sulfate (BLEO) in sterile saline. Two days before the administration of BLEO, one group received captopril in the drinking water at a concentration of 500 mg/liter. Another group received daily intraperitoneal injections of ZVAD-fmk at 1 mg per kg, in a 10% dimethylsulfoxide/phosphate-buffered saline (DMSO/PBS) vehicle. Control animals (NORM) received administrations of the vehicles only. Captopril and ZVAD-fmk administrations were continued for 14 days after instillation of BLEO, at which point all animals were sacrificed for histology and detection of collagen and DNA fragmentation in epithelial cells.

Immediately before sacrifice, animals were given intraperitoneal injections of sodium pentobarbital and the trachea was cannulated. The middle right lobe was ligated at the hilus, was excised distal to the ligation and was immediately frozen in liquid N₂ for hydroxyproline assay of total collagen (see below). The remaining lung tissues were carefully removed and were instilled with 4% paraformaldehyde in PBS at 20 cm H₂O constant pressure, then immersed in the same fixative for two hours. The fixed tissues were washed with PBS three times for 15 minutes and were then embedded in paraffin. Five-micron sections of lung were deparaffinized by passing through xylene, xylene:alcohol 1:1, 100% alcohol and 70% alcohol for 10 minutes each. Ethanol was removing by rinsing with distilled water.

In situ end-labeling (ISEL) of fragmented DNA. ISEL was conducted by a modification of the method of Mundle et al., Cell Death and Differentiation, 1: 117-122 (1994). Briefly, ethanol was removed from deparaffinized lung sections by rinsing in distilled water for at least 10 minutes. The slides were then placed in 3% hydrogen peroxide (Sigma Chemical, St. Louis, Mo.) for 30 minutes at 20° C., rinsed with PBS, and incubated with Proteinase K (Sigma Chemical, St. Louis, Mo.) in saline sodium citrate solution (SSC) (0.3 M NaCi and 30 mM sodium citrate in water, pH 7.0) for 15 minutes at 37° C. Samples were rinsed once in water, three times in 0.15 M PBS for 4 minutes each, and then incubated in SSC at 80° C. for 20 minutes. After four rinses in PBS and four rinses in Buffer A (50 mM Tris/HCl, 5 mM MgCl, 10 mM β-mercaptoethanol and 0.005% BSA in water, pH 7.5), the sections were incubated at 18° C. for two hours with ISEL solution (0.001 mM digoxigenin-dUTP, 20 U/ml DNA Polymerase 1 and 0.01 mM each of dATP, dCTP and dGTP in Buffer A). The sections were then rinsed thoroughly five times with Buffer A and three times with PBS. Detection of incorporated dUTP was achieved by incubation for two hours at 37° C. with AP-conjugated anti-digoxigenin (Boerhinger Mannheim, Indianapolis, Ind.) at a 1:400 dilution. Bound AP-antibody complexes were detected with the Fast Blue chromogen system and the sections were mounted with Fluoromount Solution (Southern Biotechnology, Birmingham, Ala.).

Identification of Collagen. Collagens were localized through staining of lung sections by the picrosirius red technique, see Pick et al., Am. J. Pathol. 134:365-371 (1989), which has absolute specificity for collagen. Briefly, sections deparaffinized with xylenes were rehydrated through a descending series of ethanols to distilled water. The hydrated sections were immersed in 0.2% aqueous phosphomolybdic acid for 2 minutes, rinsed in distilled water and then stained for 110 minutes with 0.1% Sirius Red F3BA (Pfal and Bauer, Stamford, Conn.) in saturated aqueous picric acid, pH 2.0. The sections were washed for 2 minutes in 0.01 N HCl, rinsed for 45 seconds in 70% ethanol, dehydrated in three changes of absolute alcohol, cleared in xylene and mounted in a nonaqueous mounting medium. Fibrotic foci were detected by polarization microscopy, under which collagen fibers appear yellow-white, see Uhal, et al., Am. J. Physiol. 275:L1192-L1199 (1998), or white in greyscale images. Quantitation of collagens in alveolar septa and peribronchial parenchyma by digital imaging was as described in detail below.

For quantitation of total lung collagen, tissues frozen in liquid N₂ (see above) were dried to constant weight in pre-weighed tubes at 80° C. The weighed dry tissue was hydrolyzed in 6 N HCl and was subjected to determination of hydroxyproline as described in Woessner et al., Arch. Biochem. Biophys. 93:440-447, 1961. The efficiency of the hydrolysis was verified with rat tail collagen by comparison to standard hydroxyproline (Sigma, Saint Louis, Mo.).

Lung Cell Isolation and Detection of Apoptosis In Vitro. Primary alveolar epithelial cells (AEC) were isolated from adult male Wistar rats as described above. The primary cells were studied at day two of culture, a time at which they are type II cell-like by accepted morphologic and biochemical criteria, see Paine et al., Am. J. Physiol. 270:L484-L486 (1996), and all preparations were of greater than 90% purity as assessed by acridine orange staining as discussed above. All cells were seeded in 24-well or 6-well chambers, and all experiments were conducted at subconfluent densities of 80-90% in serum-free Ham's F12 medium. Test reagents were diluted with Ham's F12 medium and were applied for 20 hours at 37° C. in a 5% CO₂ incubator.

Detection of apoptotic cells with propidium iodide (PI) was conducted as described above, following digestion of ethanol-fixed cells with DNAse-free RNAse in PBS containing 5 ug/ml PI. Cells exhibiting nuclear fragmentation and chromatin condensation (described above) were scored in four randomly selected fields per culture well in at least four culture vessels per experimental condition, and were expressed as a percentage of the total cells within the same field (minimum of 100 cells/field).

Microscopy and Image Analysis. The prepared sections were photographed under transmitted or polarized light on an Olympus BH2 epifluorescence microscope fitted with automatic photographic equipment. Color photographic slides made with tungsten film were converted to digital images by scanning, and the images were compiled with commercially available image analysis software.

For quantitation of collagen stained with picrosirius red (PR), polarized light images were converted to greyscale, and the total number of white pixels specific for collagen, see Pick, et al., Am. J. Pathol. 134:365-371 (1989), per image was determined as a percentage of the total pixel area. This procedure was applied to a total of six fields per sample, and the data were compiled for statistical analyses. Collagen in “alveolar septal” regions was defined as the average PR pixel percent in alveolar regions in low magnification (100×) microscopic fields which contained no large airways. “Peribronchial” collagen was defined as the average PR pixel percent in regions which included a large airway wall and the adjacent parenchyma detectible in a 200× field (approximately 1000 microns). Data were compiled as the mean pixel area±the standard error. Tests of statistical significance were obtained by ANOVA followed by Student-Newman-Keul's post hoc analysis.

Expression of FAS by alveolar epithelial cells is observed during bleomycin-induced fibrogenesis in mice. See Hagimoto, et al., Am. J. Respir. Cell Mol. Biol. 16:91-101 (1997). To determine whether captopril or caspase inhibitors block FAS-induced apoptosis of primary rat alveolar epithelial cells in vitro, primary alveolar epithelial cells (AEC) isolated from adult Wistar rats were exposed to recombinant human FAS ligand (50 ng/ml) for 20 h in the presence or absence of captopril (500 ng/ml) or other caspase inhibitors, i.e., ZVAD-fmk, (N-benzylcarboxy-Val-Ala-Asp-[O-Me]-CH₂F), a caspase inhibitor with selectivity toward caspases 1 and 4; DEVD-fmk (Asp-Glu-Val-Asp-[O-Me]-CH₂F), a caspase inhibitor with selectivity toward caspase 3; and YVAD-cmk (Tyr-Val-Ala-Asp-[O-Me]-CH₂Cl), a caspase inhibitor with selectivity toward caspase 1, all at 60 uM. Apoptosis was detected by quantitation of fragmented nuclei (see above).

The results indicated that captopril at 500 ng/ml and the active site-specific caspase inhibitors ZVAD-fmk, DEVD-fmk and YVAD-cmk, all at 60 uM, had comparable efficacy to inhibit apoptosis induced by recombinant FAS ligand (p<0.01 vs. controls by ANOVA and Student-Newman-Keul's test). However, direct comparisons of the ability of the three caspase inhibitors to attenuate FAS- or TNF-induced liver damage in vivo indicated that ZVAD-fmk was more potent than the other caspase inhibitors, presumably because it is more permeable to some cell membranes. See Kunstle et al., Immunol. Lett. 55:5-10 (1997). Based on this finding, ZVAD-fmk, an inhibitor of ICE-family caspases, was used in the in vivo experiments described below.

Related in vitro experiments revealed that bleomycin sulfate was also capable of inducing apoptosis in primary AEC isolated from adult Wistar rats. Primary alveolar epithelial cells (AEC) were exposed to bleomycin (50 uM) for a total of 20 h beginning after a one-hour pretreatment with captopril (500 ng/ml), ZVAD-fmk (60 uM) or aurintricarboxylic acid (10 uM). Apoptosis was detected as described above. By nuclear fragmentation assay, see above, induction of apoptosis was statistically significant at 5 uM bleomycin and was concentration-dependent.

At a concentration of 50 uM (BLEO 50), bleomycin-induced apoptosis of AEC in vitro was inhibited 76% by captopril at 500 ng/ml, consistent with the demonstration above that captopril inhibits FAS-induced apoptosis of AEC. See also Uhal. et al., Am. J. Physiol. 275:L1013-1017 (1998). Further, bleomycin-induced apoptosis of AEC in vitro was reduced 78% by the caspase inhibitor ZVAD-fmk, and was reduced 59% by the endonuclease inhibitor aurintricarboxylic acid (p<0.01 vs. BLEO 50 by ANOVA and Student-Newman-Keul's test). These findings confirm the specificity of the nuclear fragmentation assay as an unambiguous measure of apoptosis in this in vitro system.

In the in vivo model of fibrosis, histology of whole lung sections obtained from Wistar rats revealed extensive lesions in the bleomycin-treated group, especially adjacent to large airways. The lesions contained many lymphocytes, collapsed alveolar spaces and thickening of nearby alveolar septa. In both the captopril and ZVAD-fmk-treated animals, lesions were present but were much less extensive and contained fewer inflammatory cells.

Detection of fragmented DNA by in situ end-labeling (ISEL) found positive nuclei within the alveolar epithelial and airway epithelial cell populations in bleomycin-treated animals at 14 days after bleomycin administration. In the airways, ISEL-positive nuclei were often observed within “sheets” of epithelia detached from the underlying stroma. In both captopril-treated and ZVAD-fmk-treated animals, ISEL-labeled epithelial cells were rare, even within lesions. Quantitation of ISEL-positive nuclei in bleomycin plus captopril or ZVAD-fmk-treated cell populations confirmed the ability of captopril and ZVAD-fmk to reduce the frequency of epithelial DNA fragmentation. Further, the architecture of both the alveolar and airway epithelia appeared to be significantly preserved by both captopril and ZVAD-fmk, even within the small lesions present in these animals.

Experiments were conducted to identify the lung collagens present after bleomycin treatment with or without captopril or the caspase inhibitors. Animals were exposed to bleomycin, captopril, ZVAD-fmk or vehicle only. Replicate sections of rat lung used for ISEL were stained by the picrosirius red technique, see Pick, et al., Am. J. Pathol. 134:365-371 (1989), and viewed under polarized light, see Uhal, et al., Am. J. Physiol. 275:L1192-L1199 (1998), in which collagen appears white in greyscale images. Collagen was quantitated from polarized light images as the total number of white pixels per unit area within the alveolar and peribronchial lung parenchyma. At 14 days post-bleomycin treatment, collagen was also quantitated by hydroxyproline assay applied to hydrolyzed lung tissue. See Woessner, Arch. Biochem. Biophys. 93:440-447, 1961.

Less-extensive collagen accumulation in alveolar septa and peribronchial parenchyma was observed in animals treated with captopril or ZVAD-fmk. The distribution of collagens in alveolar septa of bleomycin-treated animals appeared thicker than in controls, even in regions distal to airways (p<0.01 vs. controls by ANOVA and Student-Newman-Keul's test). Quantitation of the total number of white pixels by digital imaging revealed that both captopril-treated and ZVAD-fmk-treated animals accumulated less collagen per unit tissue in these areas (p<0.01 vs. bleomycin alone). The inhibition was also observed in areas adjacent to bronchi: control lungs had a well-defined collagen matrix surrounding the bronchi, but in bleomycin-treated lungs this was the area of most severe collagen accumulation. Captopril and ZVAD-fmk-treated lungs did accumulate collagen in these regions, but the deposition was less severe and did not extend as far into the adjacent parenchyma. Scoring of picrosirius red staining by digital imaging in the peribronchial parenchyma confirmed this assessment. Quantitation of total lung collagens by hydroxyproline assay also confirmed that the administration of captopril or ZVAD-fmk inhibited lung collagen accumulation at 14 days post-bleomycin treatment (p<0.01 vs. bleomycin alone).

The results described above link the antifibrotic effect of ACE inhibitors to pulmonary epithelial cell apoptosis by the demonstration that the ACE inhibitor captopril has potent ability to abrogate FAS-induced apoptosis in human lung epithelial cells, at least in vitro. The data in this Example demonstrate that captopril, if applied to well-differentiated primary alveolar epithelial cells isolated from rats, also attenuates apoptosis induced by either FAS ligand or by bleomycin.

The fact that the primary cells studied in the in vitro experiments were isolated from the same rat strain used for the subsequent in vivo studies supports the conclusion that inhibition of apoptosis by captopril is likely to occur in the intact organism as well as in vitro. The data reported here also support that conclusion, and indicate that the inhibitory effects of captopril on apoptosis affect airway epithelial cells as well as those of the alveoli. Further, the results indicate that the caspase inhibitor ZVAD-fmk has essentially the same potency for inhibition of bleomycin-induced apoptosis in either lung epithelial cell population. Although both captopril and ZVAD-fnk were more potent at inhibiting apoptosis in alveolar versus airway epithelial cells, this result might be due to bleomycin-induced airway necrosis. The successful inhibition of lung epithelial cell apoptosis by ZVAD-fmk is significant in that the compound was administered intraperitoneally in this study, in contrast to the more common intravenous route of administration. See Kunstle et al., Immunol. Lett. 55:5-10 (1997); Yaoita et al., Circulation 97:276-81 (1998); and Rodriguez et al. J. Exp. Med. 184(5):2067-72 (1996).

The caspase inhibitor ZVAD-fmk was similar to captopril in its potency to attenuate the accumulation of lung collagen measured by either of two methods. These results support the conclusion that the ability of captopril to attenuate lung fibrogenesis is related to its ability to inhibit lung epithelial cell apoptosis. Although bleomycin is known to induce apoptosis in other lung cell populations, such as the alveolar macrophage, see Hamilton et al., Am. J. Physiol. 269:L318-L325 (1995), whether captopril or other ACE inhibitors block the death of that cell type is unknown. The caspase inhibitor ZVAD-fmk has been shown to attenuate alveolar macrophage apoptosis induced by silica. See Iyer et al., Am. J. Physiol. 273:L760-L767 (1997).

The experiments described here demonstrate that well-differentiated primary alveolar epithelial cells in vitro undergo apoptosis in response to purified FAS ligand or bleomycin, and that apoptosis in response to either inducer is inhibited by captopril or the caspase inhibitor ZVAD-fmk. In vivo administration of captopril or ZVAD-fmk abrogated bleomycin-induced apoptosis of alveolar epithelial cells and significantly inhibited the generation of fragmented DNA within airway epithelial cells. Captopril or ZVAD-fmk also significantly reduced the accumulation of lung collagens by fourteen days after bleomycin instillation. These results indicate that the ability of captopril to attenuate experimental lung fibrogenesis is related to its ability to abrogate apoptosis in lung epithelial cells.

### EXAMPLE 5

Human Lung Myofibroblasts Produce Angiotensin Peptides that Induce Alveolar Epithelial Apoptosis

Soluble factors produced by lung fibroblasts isolated from fibrotic lung (but not from normal lung) are capable of inducing apoptosis of A549 and primary alveolar epithelial cells, and alveolar epithelial apoptosis has been observed adjacent to abnormal fibroblasts in human lung. See Uhal et al., Am. J. Physiol. 269:L819-L828 (1995); Uhal, et al., Am. J. Physiol. 275:L1192-L1199 (1998). It is possible that fibroblasts within fibrotic human lung may synthesize angiotensin peptides and that these may be the factors that induce apoptosis in AEC.

Experiments were conducted to determine whether the soluble inducer of alveolar epithelial apoptosis produced by “fibrotic” human lung fibroblasts might be related to the expression of angiotensinogen by fibroblasts, a phenomenon observed in rat cardiac myocytes. See Katwa, et al., J. Mol. Cell. Cardiol. 29:1375-1386 (1997). As described below, alpha-smooth muscle actin-positive fibroblasts isolated from fibrotic human lung were found to express angiotensinogen, and that this protein and its product angiotensin II are the soluble inducers of alveolar epithelial cell apoptosis identified in earlier studies. See Uhal et al., Am. J. Physiol. 269:L819-L828 (1995).

Cultured human fibroblast strains most active in producing the apoptotic activity contain high numbers of stellate cells expressing alpha-smooth muscle actin, a myofibroblast marker. The apoptotic activity elutes from gel filtration columns only in fractions corresponding to protein. Western blotting of the protein fraction identified immunoreactive angiotensinogen, and two-step RT-PCR revealed expression of angiotensinogen by HIPF fibroblasts, but not by normal human lung fibroblasts. Specific enzyme-linked immunosorbent assays (ELISA) detected angiotensin II (ANGII) at concentrations 6-fold higher in HIPF-conditioned media than in normal fibroblast-conditioned media. Further, pretreatment of the concentrated media with purified renin plus purified angiotensin converting enzyme (ACE) increased the ELISA-detectible ANGII by 8-fold. Apoptosis of AEC in response to HIPF-conditioned media was completely abrogated by the ANGII receptor antagonist saralasin (50 ug/ml) or by anti-ANGII antibodies. These results identify angiotensinogen and its derivative angiotensin II as the protein inducers of AEC apoptosis produced by fibroblasts from fibrotic human lung. This suggests a mechanism for AEC death adjacent to myofibroblasts in fibrotic human lung.

Reagents and Materials. Purified angiotensin II, lisinopril, saralasin, purified angiotensinogen, fluorescein isothiocyanate (FITC)-conjugated antibodies to alpha-smooth muscle actin (clone 1A4) and antisera against angiotensinogen fragments were obtained from Sigma Chemical, Saint Louis, Mo. Neutralizing antibodies specific for angiotensin II were obtained from Peninsula Laboratories, San Carlos, Calif. The peptide inhibitor of Caspase 3/CPP32/YAMA, Asp-Glu-Val-Asp-fluoromethyl ketone (DEVD-fmk) was obtained from Clontech, Palo Alto, Calif. All other materials were of reagent grade, or were obtained from sources described above and in Uhal, et al., Am. J. Physiol. 275:L1192-L1199 (1998) and Uhal, et al., Am. J. Physiol. 275:L1013-1017 (1998).

Fibroblast Isolation, Culture and Preparation of Conditioned Media. Primary human lung fibroblasts were isolated at the National Institute of Respiratory Diseases, Mexico. See Uhal et al., Am. J. Physiol. 269:L819-L828 (1995). Of these, “fibrotic” lung fibroblast strains (HIPF-X, where X=patient number) were derived from patients suffering from either of two different types of interstitial lung disease (ILD): Idiopathic Pulmonary Fibrosis (IPF) and Chronic Hypersensitivity Pneumonitis (CHP). In both diseases, patients had clinical, functional and radiologic features which fulfill the diagnostic criteria for an ILD. See Selman M., Pulmonary fibrosis: Human and experimental disease in Rojkind M. (ed.) Connective Tissue in Health and Disease, CRC Press Inc., Boca Raton, Fla. (1990) at 123-188. Briefly, all patients had progressive dyspnea, bilateral reticulonodular images on chest roentgenogram, restrictive lung functional impairment with decreased lung volumes and compliance, and hypoxemia at rest that worsened with exercise. In addition, patients with CHP had domestic exposure to pigeons that predated the development of respiratory symptoms, specific serum antibodies against avian antigens determined by ELISA, and a morphological study consistent with the diagnosis. The tissue samples showed a diffuse interstitial inflammation of mononuclear predominance, mainly lymphocytes, and frequent multinucleated giant cells in terminal and respiratory bronchioles as well as in the alveolar spaces. Small and loosely arranged granulomas were observed in the interstitium, and there were no histological changes suggestive of infection or another ILD. Patients with IPF had neither antecedents of any occupational or environmental exposure nor other known cause of ILD. Morphological study showed patchy alveolar septal fibrosis and interstitial inflammation consisting mostly of mononuclear cells but also of neutrophils and eosinophils. Variable macrophage accumulation was observed in the airspaces, as was cuboidalization of the alveolar epithelium. Biopsies lacked granulomas, vasculitis, microorganisms, and inorganic material by polarized light microscopy. See Antoniades et al., J. Clin. Invest. 86:1055-1064 (1990).

Control fibroblasts were obtained from individuals having undergone a lobectomy for removal of a primary lung tumor. No morphologic evidence of disease was found in the tissue samples used for the isolation of control cells (N-X). Lung fibroblasts from normal or fibrotic tissue were isolated by trypsin dispersion, and fibroblast strains were established in Dulbecco's modified Eagle's medium (or in Ham's F-12 medium) supplemented with 10% fetal calf serum, 200 U/ml penicillin, and 200 mg/ml streptomycin. All cells were cultured at 37° C. in 95% air-5% CO₂ until early confluence. For preparation of conditioned media, fibroblasts were incubated for 24 h in serum-free Ham's F12, and the media were collected and centrifuged to remove detached cells. The cell-free conditioned media were lyophilized or used directly. Only early passage (3-12) primary cell cultures were used for the preparation of conditioned media. Essentially 100% of all fibroblast isolates were immunoreactive with monoclonal antibodies to vimentin. See Uhal et al., Am. J. Physiol. 269:L819-L828 (1995) and Uhal, et al., Am. J. Physiol. Suppl. (October) 261:110-117 (1991).

Epithelial Cell Culture and Detection of Apoptosis. Primary alveolar type II epithelial cells were isolated from adult male Wistar rats by a trypsin digestion and density gradient protocol. See Uhal et al., Am. J. Physiol. 264:L153-L159 (1993). All epithelial cell preparations were greater than 90% pure as determined by acridine orange uptake into lamellar bodies. See Uhal, et al., Am. J. Physiol. Suppl. (October) 261:110-117 (1991). Epithelial cell apoptosis was detected by the assessment, under fluorescence microscopy, of chromatin condensation and nuclear fragmentation within cytokeratin-positive cells after ethanol fixation and staining with propidium iodide. See Uhal et al., Am. J. Physiol. 269:L819-L828 (1995) and Uhal, et al., Am. J. Physiol. 275:L1013-1017 (1998). In these assays, detached cells were retained by centrifugation of the culture vessels during fixation with 70% ethanol. For testing of conditioned media, cultured epithelial cells were incubated for 20 h with each medium diluted 1:5 in serum-free Ham's F12 medium; this concentration provided a maximal response at the least expense of medium. See Uhal et al., Am. J. Physiol. 269:L819-L828 (1995). All test agents (i.e., saralasin, angiotensins) were dissolved directly in culture medium, except DEVD-fmk, which was first suspended in dimethylsulfoxide (DMSO). Control experiments showed no significant effect of the small vehicle volume on apoptosis or its inhibition.

Flow Cytometry and Microscopy. Flow cytometric analyses were performed on a Partec CA-III flow cytometer equipped with a 25 mW argon ion laser for excitation at 488 nm. Propidium iodide fluorescence data were acquired through a 610 nm long-pass filter, and fluorescein (FITC) fluorescence was acquired through an EM520 band-pass filter. Following standardization with fluorescent microspheres (Coulter Corp., Hiahlea, Fla.), amplifier gains were not changed throughout an experiment. Flow cytometric analyses of fibroblast ploidy (DNA content) versus alpha-smooth muscle actin immunoreactivity (α-SMA-FITC) were conducted as described in Uhal, et al., Am. J. Physiol. 275:L998-L1005 (1998). Briefly, cells were lightly trypsinized from the culture dishes, dispersed by agitation and fixed in 70% ethanol. The fixed cells were incubated with FITC-conjugated anti-α-SMA diluted 1:400 in 1% bovine serum albumin (BSA) in PBS, pH 7.3; the cells were washed and resuspended in PBS containing DNAse-free RNAse and 5 ug/ml propidium iodide (PI). Anti-α-SMA fluorescence and DNA content data were acquired in log and linear scale, respectively.

Protein Methods. Gel filtration was performed on lyophilized serum-free conditioned media resuspended in deionized water at 20% of its original volume. The concentrated media was loaded onto Sephadex PD-10 columns (Pharmacia Biotech, Uppsala, Sweden) which were pre-equilibrated with serum-free Ham's F12 medium, and fractions were eluted with the same F12 serum-free medium. Collected fractions were diluted 1:3 with serum-free Ham's F12 medium and were tested for apoptotic activity as described above. For Western blotting, conditioned media concentrated as just described were dialyzed against deionized water through 500 Dalton molecular-weight-cutoff membranes (Spectrum Laboratories, Houston, Tex.) until the medium phenol red was no longer visible. The dialysate was lyophilized, resuspended in gel-specific loading buffer and electrophoresed on 16% Tricine Ready-Gels (Bio-Rad, Hercules, Calif.). Medium proteins were transferred to polyvinylidenedifluoride (PVDF) membranes (BioRad) by electroblotting and were immunoblotted with rabbit antisera against angiotensin peptides (Sigma Chemical, Saint Louis, Mo.). Reactive protein was detected with secondary anti-rabbit-IgG-biotin, strepatavidin-alkaline phosphatase and a nitro blue tetrazolium chromogen.

RT-PCR. Reverse transcription-polymerase chain reaction (RT-PCR) assays of angiotensinogen gene products were performed as described below. Briefly, total RNA was isolated by the RNeasy Mini protocol (Qiagen Co., Santa Clarita, Calif.). To synthesize cDNA by RT-PCR, 3 ug of purified RNA was reverse-transcribed using 2 uM oligo-dT, 50 mM Tris-HCl (pH 8.3), 75 mM KCl, 3 mM MgCl₂, 0.01 mM DTT, 0.2 mM of each dNTP, 1 U/ul RNase inhibitor (RNasin), and 2 U of avian myoblastosis virus reverse transcriptase (AMV-RT) (Promega Co., Madison, Wis.) in a total volume of 30 ul. The reaction was performed for 1 hour at 45° C. followed by 20 cycles of PCR amplification as described below. PCR amplification was performed with 10 ul aliquots of cDNA, obtained as just described above, equivalent to 1 ug of the starting RNA. The identity of expressed genes was determined by correspondence to the expected size of the PCR product in 1.6% agarose gels.

For RT-PCR from the human fibroblast strains, the following primers were used: for angiotensinogen (ANGEN), the outermost primer pair for a two-step nested amplification consisted of a “coding strand” oligonucleotide having the sequence 5′ GCTTTCAACACCTACGTCCA 3′ [SEQ I.D. NO. ______] and a “noncoding strand” oligonucleotide having the sequence 5′ AGCTGTTGGGTAGACTCTGT 3′ [SEQ I.D. NO. ______]; the innermost primer pair for the nested amplification consisted of a “coding strand” oligonucleotide having the sequence 5′ TTCTCCCTGCTGGCCGAG 3′ [SEQ. I.D. NO. ______] and a “noncoding strand” oligonucleotide having the sequence 5′ GGGCTCTCTCTCATCCGC 3′ [SEQ. I.D. NO. ______]. These primer sets were used sequentially to amplify a final PCR product of 447 basepairs (bp). See Lai et al., J. Hypertens. 16:91-102 (1998).

For β-actin, single-step RT-PCR was used with a “coding strand” oligonucleotide having the sequence 5′ AGGCCAACCGCGAGAAGATGACC 3′ [SEQ. I.D. NO. ______] and a “noncoding strand” oligonucleotide having the sequence 5′ GAAGTCCAGGGCGACGTAGC 3′ [SEQ. I.D. NO. ______], which amplify a PCR product of 332 bp. See Ponte et al., Nucl. Acid Res. 12:1687-1696 (1984). The ANGEN:β-actin ratio was calculated from densitometric scans of PCR products detected by ethidium bromide staining of the PCR products after electrophoresis in 1.6% agarose gels.

Microscopy and image analysis. Photomicroscopy was performed on an Olympus EMT-2 epifluorescence phase-contrast microscope equipped with band-pass filters for detection of FITC and propidium iodide, respectively, and fitted with both color and greyscale CCD cameras.

Phase contrast microscopy was performed on primary human lung fibroblasts isolated from normal and fibrotic (N-11, N-13, and N-A strains) human lungs. The normal strains exhibited the expected spindle morphology and a relative paucity of inclusion bodies. Fibroblast isolates from fibrotic HIPF (Human Interstitial Pulmonary Fibrosis) human lung strains (e.g., HIPF-19, HIPF-28) displayed morphologic characteristics typical of myofibroblasts, including flattened and stellate morphology with occasional lipid inclusions. Because of size differences, the number of HIPF fibroblasts per culture vessel at early confluence was roughly one-half that observed for the normal fibroblast strains.

Flow cytometric (fluorescence-activated cell sorting, FACS) analysis of the expression of alpha-smooth muscle actin (α-SMA) indicated that most HIPF strains contained significantly higher numbers of α-SMA-positive cells than the normal strains. The bivariate analysis to determine DNA content (x-axis) versus immunoreactivity to fluorescent antibodies specific for α-SMA (y-axis) was as described in Uhal, et al., Am. J. Physiol. 275:L998-L1005 (1998). In Uhal et al., Am. J. Physiol. 269:L819-L828 (1995), all such fibroblast isolates were shown to be heavily immunoreactive with monoclonal antibodies to vimentin.

Table I below reports the flow cytometric data together with the potency of conditioned media from each strain to induce apoptosis in alveolar epithelial cells, determined as described in Uhal, et al., Am. J. Physiol. 269:L819-L828 (1995). The percentage of α-Actin positive cells was determined from same T-flask in which serum-free conditioned media were generated. The percentage increase in rat AEC apoptosis was determined by propidium iodide assay of primary alveolar epithelial cells exposed to serum-free conditioned media, see Uhal et al., Am. J. Physiol. 269:L819-L828 (1995); the reported percentage increase is relative to cells exposed to serum-free media alone. All activities of HIPF media were significantly greater than serum-free media (p<0.05).

TABLE I
Alpha-Smooth Muscle Actin Expression and Epithelial Apoptotic
Activity of Normal and Fibrotic Human Lung
Fibroblast Isolates.

| Human | α-Actin- | Apoptosis |
|---|---|---|
| Fibroblast | Positive | of Rat AEC, |
| Strain | Cells, % | % Increase |
| N-A | 10 | N.S. |
| N-11 | 9 | N.S. |
| N-13 | 5 | N.S. |
| HIPF-842 | | 311 + 17 |
| HIPF-19 | 39 | 172 + 7 |
| HIPF-28 | 22 | 291 + 5 |
| HIPF-112 | 52 | 287 + 10 |
| HIPF-61 | 5 | N.S. |

N.S. = no significant increase above the activity of serum-free media alone.

All strains with apoptotic activity also contained α-SMA-positive cells in higher proportions than did the normal isolates. Over three years of study, only one normal strain (N-12) in one passage (passage 6) exhibited modest apoptotic activity and concurrent increased expression of α-SMA; conversely, one fibrotic strain (HIPF-61) exhibited both no apoptotic activity and a low percentage of cells expressing α-SMA. The remainder of this Example will focus on the strains listed in Table I.

Gel filtration analyses of concentrated HIPF media indicated that the epithelial apoptotic activity eluted exclusively in the protein fraction. No activity was found in low-molecular weight fractions or in organic extracts of the medium.

Specific ELISA for angiotensin H (ANGII) detected the peptide at concentrations that were significantly higher in HIPF-conditioned media than in normal fibroblast media. Conditioned media from three normal (strains NA, N-11 and N-13) and three fibrotic (strains HIPF-19, HIPF-28 and HIPF-112) primary fibroblast isolates were concentrated and assayed by specific ELISA for ANGII content. Further, preincubation of the media with purified renin and purified angiotensin converting enzyme (ACE) increased the ELISA-detectible ANGII concentration by roughly eightfold (p<0.05 and p<0.01, respectively, versus corresponding normal strain value by ANOVA and Student-Newman-Keul's test), confirming that angiotensinogen is present in the conditioned media in much higher abundance than ANGII.

Media conditioned by the HIPF-8 fibroblast strain was dialyzed against water, lyophilized and electrophoresed on 16% tricine gels. The electrophoretically separated proteins were transferred to PVDF membrane and the membrane was immunoblotted with rabbit antisera against angiotensin peptides. A single major immunoreactive band was observed which corresponded to the known molecular weight of angiotensinogen (58 kDa). Although no immunoreactive bands corresponding to angiotensins I and II (1.4 and 1.0 kDa, respectively) were observed, peptides of this small size were found to have passed completely through the PVDF membrane, precluding their analysis by this assay.

Total RNA isolated from normal and fibrotic human fibroblast strains was subjected to two-step “nested” RT-PCR for angiotensinogen (ANGEN) and for β-actin as a normalization standard. The ANGEN:β-actin ratio was determined by densitometric scanning of PCR products detected with ethidium bromide after electrophoresis in 1.6% agarose gels. RT-PCR of total RNA isolated from HIPF fibroblast strains with primers specific for human angiotensinogen generated a single PCR product of 447 bp, the size expected in cells expressing the propeptide. See Lai et al., J. Hypertens. 16:91-102 (1998). By densitometry, this PCR product was roughly eight-fold more abundant in total RNA isolates from “fibrotic” HIPF fibroblast strains than in those from the normal strains.

HIPF-lung-fibroblast-conditioned media (strains HIPF-19, HIPF-28 and HIPF-112) were tested for apoptotic activity as in Uhal, et al., Am. J. Physiol. 269:L819-L828 (1995), in the presence and absence of DEVD-fmk (Asp-Glu-Val-Asp-fluoromethylketone) (60 uM), a sequence-specific inhibitor of Caspase-3; the nonselective angiotensin II receptor antagonist saralasin (50 ug/ml); rabbit IgG specific for angiotensin II (1 ug/ml), with <1% crossreactivity for angiotensin I or angiotensinogen; purified angiotensin II, 5 uM; purified angiotensinogen, 5 uM, and/or non-specific rabbit IgG.

The epithelial apoptotic activity of HIPF-conditioned media was completely abrogated by the sequence-specific peptide inhibitor of Caspase 3, DEVD-fmk (p<0.01 versus control by ANOVA and Student-Newman-Keul's test). These results are consistent with the results above showing that the endonuclease inhibitor aurintricarboxylic acid (ATA) could abrogate HIPF-induced alveolar epithelial apoptosis. See also Uhal et al., Am. J. Physiol. 269:L819-L828 (1995). These results also confirm that the bioassay employed is a reliable measure of apoptosis.

The HIPF-induced activity was eliminated by the nonselective angiotensin II receptor antagonist saralasin at a concentration of 50 ug/ml. Further, HIPF-induced apoptosis could be abrogated by antibodies specific for angiotensin II. The same antibody preparation also eliminated apoptosis induced by either purified ANGII or purified angiotensinogen, but nonspecific isotype-matched control antibody had no such effect. Bioassay of the fibroblast-conditioned media thus confirmed the identity of the HIPF-derived apoptotic activity as angiotensinogen and its product angiotensin II.

As described above, potent induction of apoptosis by angiotensin II was observed in both human and rat alveolar epithelial cells at concentrations of the peptide within the physiologically relevant range. The same experiments demonstrated that well-differentiated primary isolates of rat alveolar epithelial cells could undergo apoptosis induced by purified angiotensinogen; moreover, the angiotensinogen-induced apoptosis could be blocked by an angiotensin II receptor antagonist or angiotensin converting enzyme (ACE) inhibitors. These findings indicate that well-differentiated alveolar epithelial cells express sufficient renin-like and ACE-like activities to convert angiotensinogen and angiotensin I to ANGII, at least in the rat. Alveolar epithelial cells within the intact human lung likely possess a similar capacity.

Myofibroblasts isolated from injured rat cardiac muscle are capable of synthesizing angiotensinogen in vitro, in contrast to normal rat cardiac fibroblasts, which do not express angiotensinogen. See Katwa, et al., J. Mol. Cell. Cardiol. 29:I375-I386 (1997). The same cells also expressed ACE and cathepsin D, but not renin, suggesting a limited capacity to convert some of the synthesized angiotensinogen to the processed peptides angiotensins I and II. In the present study, the ELISA data indicate the presence of a small amount of angiotensin II in human lung fibroblast-conditioned media, but pretreatment of the media with purified converting enzymes generated significantly more ANGII. These data suggest that human lung myofibroblasts express limited enzymatic activities for angiotensinogen conversion. The identity of the converting enzymes expressed by these cells is currently unknown.

The ability of primary alveolar epithelial cells to undergo ANGII receptor-dependent apoptosis in response to purified angiotensinogen suggests that the enzymatic conversion of angiotensinogen to ANGI and ANGII by myofibroblasts may not be required for the induction of an apoptotic response in adjacent epithelial cells. In biopsy tissues from patients with advanced pulmonary fibrosis, alveolar epithelial cell apoptosis and necrosis were found immediately adjacent to foci of alpha-actin positive myofibroblasts. See Uhal, et al., Am. J. Physiol. 275:L1192-L1199 (1998). Because the biopsy specimens examined in that study were the same as those used for the isolation of the HIPF “fibrotic” fibroblast strains used in the present study, the results suggest that the production of angiotensinogen by myofibroblasts is a plausible mechanism to explain the colocalization of alveolar epithelial cell apoptosis adjacent to these foci in situ.

As discussed above, very little is known about the local concentrations of angiotensinogen or angiotensin II in extravascular compartments of the lung. In patients with Adult Respiratory Distress Syndrome, see Wenz et al., Chest 112:478-483 (1997), the mean concentration of angiotensin II in arterial plasma (85 pg/ml, or 0.1 nM) is just below the lowest concentration of angiotensin II found in the experiments described above to elicit a statistically significant induction of apoptosis in primary alveolar epithelial cells. In light of this, the local concentration of angiotensin II in the microenvironment surrounding cells which synthesize angiotensinogen is likely to exceed the threshhold necessary to induce apoptosis of adjacent epithelial cells. On the other hand, angiotensin II has a biological half-life of less than one minute, see Vernace et al., Hypertension 23:853-856 (1994), and thus the microenvironment in which apoptosis of epithelial cells might be induced would be expected to be of limited size. Such a scenario is consistent with observations of focal alveolar epithelial cell apoptosis and necrosis in the vicinity of alpha-actin-positive fibroblastic foci. See Uhal, et al., Am. J. Physiol. 275:L1192-L1199 (1998).

The induction of angiotensinogen expression has been linked to several profibrotic processes. Angiotensin II increases type I collagen synthesis in myofibroblast-like cultured valvular interstitial cells of the heart, see Katwa, et al., Cardiovasc. Res. 29:57-64 (1995), and angiotensin II receptor antagonists are known to attenuate the formation of fibrous tissue at the site of experimental myocardial infarction. See Smits et al., J. Cardiovasc. Pharmacol. 20:772-778 (1992). As discussed in Katwa, et al., J. Mol. Cell. Cardiol. 29:I375-I386 (1997), the myofibroblast is one of the key sources of angiotensinogen, and thus angiotensin II, at these sites. In the lung, the elimination of alveolar epithelial cells by angiotensin II-induced apoptosis would be expected to reduce the tonic suppression of fibroblast proliferation exerted by epithelial-derived prostaglandin E2, see Goldstein et al., J. Biol. Chem. 257:8630-8637 (1982), and to compromise the immunomodulatory functions of the intact epithelium. See Kang et al., Am. J. Resp. Cell. Mol. Biol. 9:350-355 (1993) and Crouch et al., Am. J. Respir. Cell Mol. Biol. 12:410-415 (1995). Further, the resulting loss of portions of the epithelial barrier would increase the exposure of underlying mesenchymal cells to proinflammatory cytokines released by free alveolar cells. See Simon, Lung Biology in Health and Disease 80:511-540 (1995).

In summary, primary fibroblasts isolated from fibrotic human lung synthesize angiotensinogen and limited amounts of angiotensin II. Apoptosis of cultured alveolar epithelial cells in response to serum-free media conditioned by these cells is completely abrogated by antagonists of the renin-angiotensin system and by specific antibodies to angiotensin II. These results have identified the fibroblast-derived soluble inducer of alveolar epithelial apoptosis as angiotensinogen and its product angiotensin II. See Uhal et al., Am. J. Physiol. 269:L819-L828 (1995). They also provide a mechanism for the ability of ACE inhibitors to abrogate experimental lung fibrogenesis in animal models. See Molteni, et al., Proc. Soc. Exp. Biol. Med. 180:112-120 (1985) and Ward et al., Int. J. Radiat. Oncol. Biol. Phys. 19:1405-1409 (1990).

### EXAMPLE 6

Fas-Induced Apoptosis of Alveolar Epithelial Cells Requires Angiotensin II Generation And Receptor Interaction.

Experiments were conducted to examine the possibility that the de novo synthesis of ANGII, and its subsequent binding to the ANGII receptor, might be necessary for apoptosis in response to Fas.

Reagents and Materials. Purified angiotensin II (ANGII), angiotensinogen, lisinopril, saralasin and antibodies to ANGII and angiotensinogen were obtained from Sigma Chemical Company, Saint Louis, Mo. Primers for reverse-transcription-polymerase chain reaction (RT-PCR) were synthesized by GeneMed Synthesis, San Francisco, Calif. Lipofectin reagent (Oligofectin G) was obtained from Sequitur, Inc, Natick, Mass. All other materials were from sources described earlier (Uhal, B. D., C. Gidea, R. Bargout, A. Bifero, O. Ibarra-Sunga, M. Papp, K. Flynn and G. Filippatos, Captopril inhibits apoptosis in human lung epithelial cells: a potential antifibrotic mechanism. Am. J. Physiol. 275:L1013-1017 (1998); Wang, R., A. Zagariya, O. Ibarra-Sunga, C. Gidea, E. Ang, S. Deshmukh, G. Chaudhary, J. Baraboutis, G. Filippatos and B. D. Uhal, Angiotensin II induces apoptosis in human and rat alveolar epithelial cells. Am. J. Physiol. 276:L885-L889 (1999)), or were of reagent grade.

Cell Culture and Detection of Apoptosis. The human lung adenocarcinoma cell line A549 was obtained from American Type Culture Collection and cultured in Ham's F12 medium supplemented with 10% fetal bovine serum (FBS). Primary alveolar epithelial cells were isolated from adult male Wistar rats as described earlier (Uhal, B. D., I. Joshi, A. True, S. Mundle, A. Raza, A. Pardo and M. Selman. Fibroblasts isolated after fibrotic lung injury induce apoptosis of alveolar epithelial cells in vitro. Am. J. Physiol. 269:L819-L828, 1995); Wang, R., A. Zagariya, 0. Ibarra-Sunga, C. Gidea, E. Ang, S. Deshmukh, G. Chaudhary, J. Baraboutis, G. Filippatos and B. D. Uhal, Angiotensin II induces apoptosis in human and rat alveolar epithelial cells. Am. J. Physiol. 276:L885-L889 (1999)). The primary cells were studied at day two of culture, a time at which they are type II cell-like by accepted morphologic and biochemical criteria (Uhal, B. D. Cell cycle kinetics in the alveolar epithelium. Am. J. Physiol. 272:L1031-L1045 (1997)), and all preparations were of greater than 90% purity assessed by acridine orange staining as discussed previously (Uhal, B. D., K. M. Flowers and D. E. Rannels, Type II pneumocyte proliferation in vitro: problems and future directions. Am. J. Physiol. Suppl. (October) 261:110-117 (1991)). All cells were seeded in 24-well or 6-well chambers, and all experiments were conducted at subconfluent densities of 80-90% in serum-free Ham's F12 medium. Test reagents were diluted with serum-free Ham's F12 medium and were applied under serum-free conditions for 20 hours at 37C. in a 5% CO2 incubator. Detection of apoptotic cells with propidium iodide (PI) was conducted as described above Am. J. Physiol. 275:L1013-1017 (1998); Uhal et al., following digestion of ethanol-fixed cells with DNAse-free RNAse in PBS containing 5 μg/ml PI. In all assays, detached cells were retained by centrifugation of the culture vessels during fixation with 70% ethanol, or by retention of culture media and recovery by centrifugation before assay. As in the above Examples, the induction of apoptosis was verified by Annexin V binding and generation of DNA strand breaks

RT-PCR. Reverse transcriptase polymerase chain reaction (RT-PCR) assay of angiotensinogen gene products was performed as described in Wang et al., Am. J. Physiol. 276:L885-L889 (1999) in a total reaction volume of 30 μl. Total RNA was isolated by the RNeasy Mini protocol (Qiagen Co., Santa Clarita, Calif.). The reaction was performed for 1 hour at 45 C. followed by 20 cycles of PCR amplification. PCR amplification was performed with 10 ul aliquots of cDNA, equivalent to 1 ug of the starting RNA. The identity of expressed genes was determined by expected size of the PCR product in 1.6% agarose gels.

For RT-PCR from the human A549 cells, the following primers were used: for angiotensinogen, “outer” primer of a two-step nested assay, coding=5′ GCTTTC-AACACCTACGTCCA 3′ [SEQ. ID NO. ______], and noncoding=5′ AGCTGTTGGGTAGACTCTGT 3′ [SEQ. ID. NO. ______]; for the “inner” primer of the nested assay, coding=5′ TTCTCCCTGCTGGCCGAG 3′ [SEQ. ID NO. ______], and noncoding=5′ GGGCTCTCTCTCATCCGC 3′ [SEQ. ID NO. ______]. These primers yield a final PCR product of 447 bp (8). For β-actin, single-step RT-PCR was used with the primers: coding=5′ AGGCCAACCGCGAGAAGATGACC 3′ [SEQ. ID NO. ______], and noncoding=5′ GAAGTCCAGGGCGACGTAGC 3′ [SEQ. ID NO. ______], which produces a PCR product of 332 bp (Ponte, P., S. Y. Ng, J. Engel, P. Gunning and L. Kedes, Evolutionary conservation is in the untranslated regions of actin mRNAs: DNA sequence of a human b-actin cDNA. Nuc. Acid Res. 12:1687-1696 (1984)).

For RT-PCR of rat-specific gene products, the following primers were used: for angiotensinogen, coding=5′ CCTCGCTCTCTGGACTTATC 3′, and noncoding=5′ CAGACACTGAGGTGCTGTTG 3′, which yields a PCR product of 226 bp by single-step RT-PCR (Pierzchalski et al., Exp. Cell Res. 234:57-65 (1997)). For β-microglobulin, the primers used were: coding=5′ CTCCCCAAATTCAAGTGTACTCTCG 3′, and noncoding=5′ GAGTGACGTGTTTAACTCTGCAAGC 3′, which yields a product of 249 bp (Katwa et al., Cardiovasc. Res. 29:57-64 (1995)).

Antisense Transfection and In Situ Hybridization. Phosphorothioated control and antisense oligonucleotides for human angiotensinogen (18-mers) were synthesized and transfected into A549 cells using the lipofectin reagent Oligofectin G (Sequitur, Inc., Natick, Mass.) as the vehicle diluted in cell culture medium. The control nucleotides were of the same length and base composition as the antisense, but with scrambled sequence. The oligonucleotide:lipofectin ratio was optimized (over a 4 h tranfection) to yield transfection efficiencies of 50-75% with no apparent cell loss or detachment. Transfection efficiency was monitored with FITC-labeled 25-mer oligonucleotide for luciferase (not shown). Transfections were conducted for 4 hours followed by 5-fold washing with serum-free cell culture medium; immediately thereafter, Fas activator or vehicle was applied as described above for 20 h. The transfection protocol itself had no significant effect on basal or Fas-induced apoptosis (not shown). Phosphorothioated oligonucleotides used for transfection were: (ANGEN antisense) 5′ CCGTGGGAGTCATCACGG 3′ [SEQ. ID NO. ______], and (ANGEN scramble) 5′ CAGGGATCTCTGGCGGAC 3′ [SEQ. ID NO. ______] as described by Phillips et al. Kidney International 46:1554-1556 (1994).

In situ hybridization was performed essentially as described by Panoskaltsis-Mortari et al., Biotechniques 18:300-307 (1995), except that no 80° C. denaturation step was employed because single-stranded probes were used. Fixed A549 cells were hybridized with digoxigenin-labeled antisense and control oligonucleotide DNA probes specific for ANGEN, which were detected with an amplified biotin/avidin system linked to nitro blue tetrazolium chromogen (purple is positive). As in the tranfection experiments, the control probes were of the same length and base composition as the antisense, but with scrambled sequence. The digoxigenin-labeled probes used were: (antisense) 5′ AGGGTGGGGGAGGTGCTGAACAGC 3′ [SEQ. ID NO. ______] and (scrambled) 5′ GATGGGGGTGGGGGACCGTAGCAA 3′ [SEQ. ID NO. ______], as described by Lai et al., J. Hypertens. 16:91-102 (1998).

The results show that induction of angiotensinogen expression, its proteolytic processing, and the subsequent binding of angiotensin II to its receptor are required events in the signaling of AEC apoptosis by Fas. Apoptosis was found to be induced in the AEC-derived human lung carcinoma cell line A549 or in primary AEC isolated from adult rats, respectively, by receptor-activating anti-Fas antibodies or by purified recombinant Fas ligand. Apoptosis in response to either Fas activator was inhibited in a dose-dependent manner by the non-thiol ACE inhibitor lisinopril or by the nonselective ANGII receptor antagonist saralasin, with maximal inhibitions of 82% and 93% at doses of 0.5 and 5 μg/ml, respectively.

In both cell types, activation of Fas caused a significant increase in the abundance of mRNA for angiotensinogen (ANGEN) that was unaffected by saralasin. Transfection of A549 cells with antisense oligonucleotides against ANGEN mRNA inhibited the subsequent induction of Fas-stimulated apoptosis by 70% (p<0.01). Activation of Fas increased the concentration of ANGII in the serum-free extracellular medium by 3-fold in primary AEC and by 10-fold in A549 cells. Apoptosis in response to either Fas activator was completely abrogated by neutralizing antibodies specific for ANGII (p<0.01), but isotype-matched nonimmune immunoglobulins had no significant effect. These results indicate that the induction of AEC apoptosis by Fas requires a functional renin-angiotensin system (RAS) in the target cell. They also indicate that therapeutic control of AEC apoptosis is possible through pharmacologic manipulation of the local RAS.

Fas-induced apoptosis in cultured alveolar epithelial cells was abrogated by angiotensin converting enzyme (ACE) inhibitors or by an angiotensin II receptor antagonist. A human lung epithelial cell line (A549) or primary isolates of rat alveolar epithelial cells (AEC) were exposed to receptor-activating anti-Fas antibody (clone CH-11) or to recombinant human Fas ligand (FASL), respectively, for 20 hours (see Uhal et al., Am. J. Physiol. 275:L1013-1017 (1998)). Apoptosis was detected by nuclear fragmentation as described above. Fas-induced apoptosis of A549 cells was significantly inhibited by the nonthiol ACE inhibitor lisinopril (500 ng/ml), and was essentially abrogated by the nonselective ANGII receptor antagonist saralasin (maximal inhibition of 93% at 50 μg/ml). In agreement with the data from A549 cells, apoptosis within primary cultures of rat AEC (induced by purified recombinant Fas ligand) also was blocked by saralasin, or by captopril or lisinopril, both at 500 ng/ml.

These findings suggested that Fas-induced apoptosis of AEC might be dependent upon de novo synthesis of ANGII and its binding to one of its receptors. To confirm this, total RNA was isolated from human A549 cells or primary rat AEC that were unactivated (CTL) or activated with Fas-ligating CH-11 antibody or purified Fas ligand in the presence or absence of saralasin (SAR). Quantitative RT-PCR was performed with primers specific for human or rat angiotensinogen (ANGEN), β-actin or β-microglobulin (b-MG). Quantitative RT-PCR revealed that activation of Fas in either A549 cells or in primary rat AEC causes significant increases in the abundance of mRNA for angiotensinogen (ANGEN), but not in the control transcripts β-actin or β-microglobulin (β-MG). In both cell types, the presence of saralasin during the Fas activation (SAR) had no effect on the accumulation of ANGEN mRNA.

To determine whether antisense oligonucleotides against angiotensinogen mRNA can inhibit Fas-induced apoptosis in A549 cells, cells were exposed for 4 h to lipofectin transfection reagent (+LIPO) in the presence or absence of antisense oligonucleotides to angiotensinogen (ANTISENSE) or control oligonucleotides of the same length and composition but with scrambled sequence (SCRAMBLE). The cells were then washed and over the subsequent 20 h, apoptosis was induced by activating anti-Fas antibody (FASmAB) and was detected as described above. A 4-hour transient transfection of antisense oligonucleotides against angiotensinogen (ANTISENSE) significantly inhibited Fas-induced apoptosis of A549 cells over the subsequent 20 hours post-transfection (70% inhibition, p<0.01). In contrast, transfection of control oligonucleotides of the same size but with scrambled sequence (SCRAMBLE) gave no inhibition. The lipofectamine transfection itself had no significant effect on either basal or Fas-induced apoptosis (not shown). To detect angiotensinogen mRNA in A549 cells, the cells were incubated with activating anti-Fas antibody (+FAS) as described above and were fixed for optimal recovery of RNA. Non-isotopic in situ hybridization was performed with oligonucleotides specific for angiotensinogen mRNA (ANGEN ANTISENSE) or with control oligonucleotides of the same length and composition but with scrambled sequence (ANGEN SCRAMBLE). When related antisense oligonucleotides against ANGEN were used for in situ hybridization, intense positive signal was found within cells that contained the fragmented nuclei typical of apoptotic cells. No positive signal was found in cells hybridized with control oligonucleotides (SCRAMBLE).

Primary isolates of rat alveolar epithelial cells (AEC) or human lung A549 cells were activated with Fas ligand (FASL) or anti-Fas antibody (FASmAB) as described above. Twenty hours later, the cell culture medium was collected, freed of detached cells, lyophilized and analyzed by enzyme-linked immunoassay (ELISA) specific for angiotensin II (ANGII). This revealed that activation of Fas significantly increased the concentration of free ANGII peptide in the culture medium bathing primary AEC or A549 cells.

Neutralizing antibodies specific for angiotensin II (ANGII) was found to abrogate Fas-induced apoptosis of AEC. Primary rat AEC or human A549 cells were made apoptotic with Fas ligand, Fas-activating antibody (FASmAB), or purified angiotensin II (ANGII) in the presence or absence of saralasin (SARAL), the caspase inhibitor ZVADfmk (Uhal et al., Am. J. Physiol.275:L1013-1017 (1998)) or neutralizing polyclonal IgG antibodies specific for ANGII (anti-ANGII). Control antibodies were non-specific isotype-matched IgGs (N.S.IgG) applied at the same concentration as anti-ANGII. These studies showed that Fas-induced apoptosis of either the rat AEC or A549 cells could be completely abrogated by neutralizing antibodies specific for ANGII (anti-ANGII). The specificity of this experimental approach is supported by the finding that the same antibody completely blocked AEC apoptosis induced by purified ANGII, whereas isotype-matched nonimmune IgGs (N.S.IgG) had no significant effects. The reliability of the nuclear fragmentation assay of apoptosis is shown by the ability of the broad-spectrum caspase inhibitor ZVADfmk (see above) to abrogate the effect of Fas ligand.

As shown above, the angiotensin I-converting enzyme (ACE) inhibitor captopril abrogated Fas-induced apoptosis in A549 cells at doses of the drug readily attained in vivo. Because captopril is a sulfhydryl-containing compound, inhibition of apoptosis was speculated to occur through thiol-mediated blockade of cysteine protease (caspase) activities required for AEC apoptosis. However, subsequent studies described herein showed that purified angiotensin II (ANGII) is capable of inducing ANGII receptor-dependent apoptosis in both A549 cells and in primary alveolar epithelial cells (AEC) isolated from rats. More importantly, the experiments reported in this Example show that the enzymatic activity of ACE and the ability of ANGII to interact with its receptor are not only involved in the signaling of Fas-induced apoptosis, but also are required for its execution.

The notion that ANGII binding to its receptor is a required step in the signaling pathway activated by Fas is supported by the ability of the nonselective receptor antagonist (saralasin) and neutralizing anti-ANGII antibodies to completely abrogate (93 and 101% inhibition, respectively) AEC apoptosis in response to Fas. Although the inhibitory actions of lisinopril and captopril on AEC apoptosis were slightly less complete (75-85% inhibition) than that of saralasin, these data do not lessen the importance of ANGII function because ACE, the preferred target of these inhibitors is only one of several dipeptidyl carboxypeptidases capable of cleaving angiotensin I (ANGI) to ANGII. Although the large degree of inhibition exerted by these compounds suggests that ACE may be the primary ANGI cleavage activity expressed by these cells, other enzymes such as chymase or cathepsins (which are not sensitive to ACE inhibitors) might also be present, and thus would preclude complete blockage of ANGII formation by these drugs.

The large increase in ANGEN mRNA in response to Fas activation (48-fold for A549 cells and 8-fold for rat AEC) suggests a mechanism in which Fas induces the transcription of ANGEN. Regardless of whether the increase in ANGEN mRNA is due to transcriptional activation or changes in transcript stability, the finding that ANGEN antisense oligonucleotides can inhibit apoptosis in response to Fas suggests that the level of functional ANGEN mRNA is critical to the process by which Fas signals AEC apoptosis. The fact that the antisense inhibition of apoptosis was not complete (70%) might be explained by the fact that the transfection efficiency was at best 75%, and thus a fraction of the cells undergoing apoptosis may not have been subject to antisense inhibition.

Regardless, the ANGEN promoter is known to contain an acute phase response (APR) element and to be rapidly responsive to cytokines such as TNF-alpha (Bardales et al., Am. J. Pathol. 149:845-852 (1996)). However, this is may be the first report of induction of ANGEN expression specifically by Fas. In cardiac myocytes, apoptosis induced by mechanical strain (“stretch”) was recently shown to be mediated by a mechanism in which increased levels of p53 protein interact directly with the ANGEN promoter to induce its transcription (Leri et al., J. Clin. Invest. 101(7):1326-42 (1998); Pierzchalski et al., Exp. Cell Res. 234:57-65 (1997)); the newly synthesized ANGEN protein is then proteolytic cleaved to ANGII, which induces the cardiomyocyte apoptosis through binding to its receptor in a manner shown herein to be analogous to that shown for AEC. In some cell types, apoptosis in response to Fas is believed to involve the induction of p53 expression (O'Conner et al., Science. 284:1431-1433 (1999)), which raises the interesting possibility that Fas activation in AEC might also result in elevated p53 protein. The possibility that the Fas-induced increase in the abundance of ANGEN mRNA is due to p53-mediated transcriptional activation is currently under investigation.

In summary, Fas-induced apoptosis of A549 cells or primary cultures of rat alveolar epithelial cells was significantly inhibited by nonthiol ACE inhibitors, and was completely abrogated by an angiotensin II receptor antagonist. Activation of Fas significantly increased the abundance of angiotensinogen mRNA, and transfection of antisense oligonucleotides against angiotensinogen significantly inhibited Fas-induced apoptosis. Activation of Fas increased the amount of free angiotensin II in the extracellular space, and neutralizing antibodies specific for angiotensin II completely blocked the induction of apoptosis by Fas. Together, these data indicate that Fas-induced apoptosis by alveolar epithelial cells requires the induction of angiotensinogen expression, its proteolytic processing, and the subsequent binding of angiotensin II to at least one of its receptors. They also indicate that therapeutic manipulation of Fas-induced apoptosis in vivo is possible with a variety of well-characterized pharmacologic antagonists of the renin-angiotensin system.

### EXAMPLE 7

ACE Inhibitors and AT1-Selective Ang Receptor Antagonists Block Amiodarone-Induced Apoptosis of Human and Rat Alveolar Epithelial Cells

The widespread use of amiodarone (AM) for the treatment of ventricular and supraventricular cardiac arrhythmias has led to increased interest in its side effects, particularly pulmonary toxicity. It is now known that pulmonary toxicity is the most significant, most limiting and potentially life-threatening side effect associated with AM use. It has been shown previously that amiodarone and its active metabolite desethylamiodarone (DES) are directly cytotoxic to cell types other than alveolar epithelial cells (AEC), such as bovine arterial endothelial cells, alveolar macrophages, human pulmonary artery endothelial cells, interstitial lung fibroblasts, bronchial epithelial cells and hepatocytes

Several mechanisms underlying the adverse pulmonary effects of amiodarone have been proposed. In addition to the direct cellular damage referred to above, these include derarrangements in lipid metabolism associated with the induction of phospholipidosis, immune-mediated mechanisms such as the activation of natural killer cell activity and the development of edema associated with an increased production of superoxide anion. The tissue level of reduced glutathione was also found to be increased significantly. In contrast, pretreatment with antioxidant agents such as butylated hydroxyanisole, vitamin E, N-acetylcysteine or ventilation with 40% oxygen protected against amiodarone-induced edema. However, the relationship of these proposed mechanisms to the induction of lung fibrogenesis is unclear.

Experiments were conducted to examine whether AM and its primary metabolite DES might be potently cytotoxic for AEC by mechanisms involving both necrosis and apoptosis. The results indicate that AM or DES induces apoptosis and necrosis of both primary rat AEC and a human AEC-derived cell line, and do so in primary AEC at concentrations significantly lower than those known be cytotoxic for other pulmonary cell types. The results also show that a significant component of the cytotoxicity is inhibitable by antagonists of the renin-angiotensin system.

The human lung adenocarcinoma cell line A549 (ATCC) was cultured in Ham's F12 medium supplemented with 10% fetal bovine serum (FBS). Primary alveolar epithelial cells were isolated from adult male Wistar rats. The primary cells were studied at day two of culture, a time at which they are type II cell-like by accepted morphologic and biochemical criteria. All primary cell preparations were of better than 90% purity assessed by acridine orange staining. All cells were seeded in 24-well or 6-well chambers, and all experiments were conducted at subconfluent densities of 80-90% in serum-free Ham's F12 medium. Test reagents were diluted with Ham's F12 medium and were applied for 20 hours at 37C. in a 5% CO₂ incubator.

Apoptotic cells were detected using propidium iodide (PI) following digestion of ethanol-fixed cells with DNAse-free RNAse in PBS containing 5 ug/ml PI. Cells with discrete nuclear fragments containing condensed chromatin were scored as apoptotic; in the primary rat AEC preparations, apoptotic cells were scored only if they also displayed immunoreactivity with anticytokeratin antibodies but not the spindle shape of fibroblasts. In replicate culture vessels, necrosis was detected as the loss of exclusion of PI after application of the vital dye directly to the cell culture medium at 5 ug/ml final concentration. Red fluorescent (PI-POSITIVE) cells (>570 nm) were scored over the subsequent hour as a percentage of the total cells in any given field. A minimum of 4 separate fields were scored in each of three separate culture vessels per experiment condition; all experiments were performed at least twice and the data were compiled. Although all necrotic cells are PI-positive, some apoptotic cells may also take up the dye but these comprise a minor fraction of the total dead cell population.

In all assays, detached cells were retained by centrifugation of the culture vessel at 300×g for 10 minutes with a centrifuge rotor designed for culture plates. For detection of necrosis (dye exclusion of PI), the PI was added directly to the cell culture medium immediately before centrifugation of the culture vessel. For detection of apoptosis, ethanol was added directly to the cell culture medium to a final concentration of 70% immediately before the centrifugation. Afterward, the ethanol was carefully removed and the total cell population (attached plus sedimented detached cells) were stained with PI as described above.

By dye exclusion assay, amiodarone (AM, Wyeth-Ayerst Research) and desethylamiodarone (DES, Wyeth-Ayerst Research) caused concentration-dependent necrosis of human A549 cells beginning at 10 ug/ml and 5 ug/ml, respectively. The same agents applied to primary cultures of rat AEC caused significant cell death beginning at 2.5 ug/ml and 0.1 ug/ml, respectively (both p<0.05), concentrations at or below the known therapeutic serum concentration of AM in patients receiving the drug for nine months. At the higher doses of DES, the cytotoxicity was essentially 100%.

AM and DES also induced apoptosis in A549 cells beginning at 10 ug/ml and 5 ug/ml, respectively, with the increase in apoptotic cells reaching a maximum of 10-fold at a dose of 10 ug/ml AM and 7-fold at a dose of 5 ug/ml DES. At doses of DES higher than 5 ug/ml, the A549 cell number remaining on the culture vessels was so low as to preclude the accurate scoring of apoptotic cells. In the primary AEC, AM and DES induced significant apoptosis beginning at 2.5 ug/ml for each agent (p<0.05), a concentration essentially the same as the therapeutic serum level of 1.8 ug/ml. The induction of apoptosis was 16-fold at 15 ug/ml AM and 3-fold at 5 ug/ml DES. In both cell types, the concentration-dependence of the induction of apoptosis was bimodal; fewer apoptotic cells were observed at the highest drug concentrations at which necrosis was overwhelmingly high.

Scoring of the total cell number at the end of the 20 hour incubations revealed that both drugs caused significant net cell loss per culture vessel in either A549 or primary AEC cultures (net cell loss defined as the decrease in attached+detached cell number). Importantly, unattached cells were recovered and included in the analyses, and thus the data represent true loss of cells rather than simple detachment from the substratum. In cultures that were not incubated with drug, the cell number did not change significantly during the 20 hour incubation.

In both A549 cells and primary rat AEC, apoptosis in response to amiodarone was inhibited by 81% and 97%, respectively, by the broad-spectrum caspase inhibitor ZVAD-fmk, confirming the specificity of the nuclear fragmentation assay for apoptosis under the conditions employed. More importantly, apoptosis of both cell types also was inhibited by the angiotensin converting enzyme inhibitor captopril (500 ng/ml, Sigma) or by the nonselective angiotensin II receptor antagonist saralasin (50 ug/ml, Sigma); these data are consistent with the earlier demonstration that captopril could block Fas-induced apoptosis of the same cells. Moreover, the same three agents significantly inhibited the amiodarone-induced decrease in total cell number of either cell type. The caspase inhibitor ZVADfmk abrogated 46% and 75%, respectively, of the net cell loss by A549 or primary rat AECs, indicating that a significant component of the cell loss was due specifically to apoptosis rather than necrosis.

Importantly, the apoptosis induced by AM could be completely blocked in either test cell type by the angiotensin receptor subtype AT1-selective antagonist Losartan (COZAAR) at a concentration of 5 pg/ml. The same AT1 antagonists also abrogated apoptosis of primary rat AECs in response to purified ANGII, purified Fas ligand or purified TNF-α. The data suggest that AT1 is the active angiotensin receptor subtype in AEC for the induction of apoptosis, regardless of whether the apoptotic stimulus is ANGII, Fas ligand, TNF-α or amiodarone. Consistent with that hypothesis, apoptosis of primary AECs in response to purified ANGII or purified amiodarone (2.5 ug/ml) was significantly inhibited by the protein kinase C (PKC) inhibitor chelerythrine, but not by the protein tyrosine phosphatase inhibitor sodium orthovanadate. These data also demonstrate that AT1 is the active ANG receptor subtype for AEC apoptosis, because downstream signaling by receptor AT1 is known to be mediated by PKC, whereas downstream signaling by receptor AT2 is mediated by protein tyrosine phosphatases. In light of this information, any AT1-selective receptor antagonist would be expected to block AEC apoptosis in response to ANGII, Fas ligand, TNF-alpha or amiodarone.

These results show that AM and DES induce apoptosis in A549 cells and in primary rat type II cells, two models of the type II alveolar epithelial cells (AEC) critical to a variety of important lung functions. The concept that AEC integrity and the ability to repair damage are critical determinants in the pathophysiology of pulmonary fibrosis is well supported. Statistically significant cell death began at 10 ug/ml for AM and at 5 ug/ml for DES in A549 cells, consistent with earlier observation that DES is more cytotoxic than AM. The well-differentiated primary AEC, however, were much more sensitive to either agent, with toxicity beginning at 1 and 0.1 ug/ml in these cells. These doses are significantly lower than those shown earlier to induce cytotoxicity in other lung cell types. Interestingly, AM and DES had equal potency for induction of apoptosis in the primary AEC, but DES was more toxic for induction of necrosis. This discrepancy might be explained by the expression of the cytochrome P450 monooxygenase system by AEC, which is believed to metabolize AM to its more toxic metabolite DES. Two types of pneumocytes in vivo exhibit substantial P450-dependant monooxygenase activity, the alveolar type II epithelial cells and the bronchiolar nonciliated Clara cells. A549 cells are not believed to express these enzymes, and this may explain the observation that A549 cells are less sensitive to AM-induced toxicity than primary AEC.

Regardless, the observation that the total cell number of either cell culture model was significantly decreased by 20 hours exposure to AM or DES indicates that these agents cause significant and net cell loss over time (rather than simple detachment, see above), despite that fact that the apoptotic indexes under the same conditions were seemingly low (5-15%). Moreover, the fact that the net cell loss was inhibitable by the caspase inhibitor ZVADfmk indicates that roughly 50% and 75% (in A549s and primary AEC, respectively) of the net cell loss could be attributed specifically to apoptosis. In vivo, the normally quiescent alveolar epithelial type II cells have an extremely high capacity to enter the cell cycle and proliferate in response to injury, but in vitro do not proliferate to any measurable degree. Thus, AM and DES may induce chronic apoptosis of AEC in patients receiving amiodarone, even at therapeutic serum levels of the drug, that might be offset by increased type II cell proliferation. As discussed earlier, the notion that ongoing apoptosis and proliferation can occur simultaneously in the alveolar epithelial cell population is supported by the observation of both a high cell birth rate and a high rate of cell death by apoptosis within the same microenvironments of lung carcinomas. Considered in this perspective, amiodarone toxicity for the lung epithelium might be viewed as dependent upon the capacity of the epithelium to offset ongoing apoptosis. Although no previous studies of amiodarone pulmonary toxicity have reported apoptosis in any lung cell type, the relatively small percentages of cells undergoing apoptosis in vitro (5-15%) support the speculation that apoptotic cells in vivo might have gone unnoticed.

More importantly, both the induction of apoptosis and the decrease in total cell number in response to amiodarone in vitro were blocked by the angiotensin converting enzyme (ACE) inhibitor captopril or by the angiotensin II receptor antagonist saralasin. Moreover, blockade by the protein kinase C (PKC) inhibitor chelerythrine but not by vanadium salts (tyrosine phosphatase inhibitors) indicates that the active angiotensin receptor is AT1, the subtype inhibitable by the antagonists Losartan (COZAAR), irbesartan and related AT1 antagonists. Recently published data indicate that Fas-induced apoptosis in A549 cells or in primary rat AEC also is potently inhibited by captopril, saralasin or other antagonists of the renin/angiotensin system, and requires the synthesis of angiotensin II (ANGII) for its execution. In light of those findings, the data in this example indicate that AEC apoptosis induced by amiodarone also requires the synthesis and binding of ANGII to its receptor, but may have no relationship to Fas or other cell surface receptors per se. Lung phospholipidosis involving surfactant-like lipids is a well-documented feature of amiodarone-induced pulmonary toxicity, but whether this phenomenon is related to dysfunction of the surfactant-producing type II epithelial cells is unclear.

The inhibition of AM-induced apoptosis and cell loss by an ACE inhibitor or an ANGII receptor antagonist (AT1-selective) is particularly interesting with regard to the widespread use and proven efficacy of these agents for a variety of thoracic and vascular diseases.

In summary, amiodarone or desethylamiodarone caused dose-dependent apoptosis, necrosis and net cell loss by human A549 cells and primary rat alveolar epithelial cells in vitro. In this cell type, the cytotoxicity was significant at or below therapeutic serum amiodarone concentrations and was severe at amiodarone concentrations known to accumulate in human lung tissue. Apoptosis and net cell loss in vitro were inhibited by the caspase inhibitor ZVAD-fmk, by the ACE inhibitor captopril or by the angiotensin II receptor antagonists saralasin or losartan (AT1-selective). The protective effect of renin-angiotensin system antagonists on amiodarone-induced apoptosis by these cells in vitro indicates that some aspects of amiodarone-induced pulmonary toxicity, particularly lung fibrosis, will be reduced by the administration of these agents concurrently.

Numerous modifications and variations of the above-described invention are expected to occur to those of skill in the art. Accordingly, only such limitations as appear in the appended claims should be placed thereon.

What is claimed is:

1. A method of treating a pulmonary fibrotic disease comprising administering to a subject at risk of or suffering from a pulmonary fibrotic disease an amount of an antagonist of the renin-angiotensin-aldosterone system effective to inhibit pulmonary epithelial cell apoptosis, wherein said antagonist is not a thiol ACE inhibitor.

2. The method of claim 1 wherein the subject is at risk of pulmonary fibrotic disease and is undergoing radiation therapy or chemotherapy.

3. The method of claim 1 wherein the subject is suffering from idiopathic pulmonary fibrosis, sarcoidosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, or drug-induced interstitial lung disease.

4. The method of claim 1 wherein said antagonist is an anti-angiotensin II antibody.

5. The method of claim 1 wherein said antagonist is an angiotensin II receptor antagonist.

6. The method of claim 5 wherein said angiotensin II receptor antagonist is selected from the group consisting of saralasin, losartan and L158809.

7. The method of claim 1 wherein said antagonist is an aldosterone antagonist.

8. A method of treating a pulmonary fibrotic disease comprising administering to a subject at risk of or suffering from a pulmonary fibrotic disease a pharmaceutically effective amount of an antagonist of the renin-angiotensin-aldosterone system, wherein said antagonist is not a thiol ACE inhibitor.

9. A method of treating a pulmonary fibrotic disease comprising administering to a subject at risk of or suffering from a pulmonary fibrotic disease a pharmaceutically effective amount of a caspase enzyme inhibitor or endonuclease inhibitor.

10. The method of claim 9 wherein said caspase enzyme inhibitor is selected from the group consisting of ZVAD-fmk, DEVD-fmk, or YVAD-cmk.

11. The method of claim 9 wherein said endonuclease inhibitor is aurintricarboxylic acid.

12. A method of identifying a novel therapeutic compound for treating pulmonary fibrotic disease comprising the steps of:
(a) measuring apoptosis of mammalian pulmonary epithelial cells in the presence and absence of a test compound;
(b) selecting a test compound that inhibits apoptosis of said cells; and
(c) determining an effect of said test compound selected in step (b) in an animal model of pulmonary fibrosis.

13. The method of claim 12 wherein in step (a) apoptosis is measured by a method selected from the group consisting of measuring in situ end-labeling of fragmented DNA, detecting nuclear and chromatin morphology, measuring Caspase-1 activity, measuring Caspase-3 activity and measuring binding of Annexin-V.

14. The method of claim 12 wherein in step (c) the animal model of pulmonary fibrosis involves administration to an animal of a toxic agent selected from the group of bleomycin, monocrotaline and Fas antibody.

15. The method of claim 12 wherein in step (c) the animal model of pulmonary fibrosis involves irradiation of an animal

16. A method of identifying a novel therapeutic compound for treating pulmonary fibrotic disease comprising the steps of:
(a) measuring the interaction of a test compound with a member of the renin-angiotensin-aldosterone system, wherein said member of the renin-angiotensin-aldosterone system is other than angiotensin converting enzyme (ACE);
(b) selecting a test compound that interacts with said member of the renin-angiotensin-aldosterone system; and
(c) determining effect of the test compound selected in step (b) in an animal model of pulmonary fibrosis.

17. The method of claim 16 wherein in step (a) wherein binding of said test compound to angiotensin II is measured.

18. The method of claim 16 wherein in step (a) wherein binding of said test compound to an angiotensin II receptor is measured.

19. A method of identifying a novel therapeutic compound for treating pulmonary fibrotic disease comprising the steps of:
(a) measuring the activity of angiotensin converting enzyme (ACE) in the presence and absence of a test compound;
(b) selecting a test compound that inhibits ACE; and
(c) measuring apoptosis of mammalian pulmonary epithelial cells in the presence and absence of said test compound selected in step (b).

20. A method of treating a human subject in need thereof comprising the steps of administering a dose of an amiodarone product higher than an amiodarone-equivalent dose of 1600 mg/day and administering an antagonist of the renin-angiotensin-aldosterone system or an apoptosis inhibitor.

21. The method of claim 20 wherein said amiodarone product is an amiodarone analog, derivative or metabolite.

22. A method of treating a human subject in need thereof comprising the steps of administering an amiodarone product at doses maintaining an amiodarone-equivalent mean serum concentration of higher than about 1.8 μg/mL and administering an antagonist of the renin-angiotensin-aldosterone system or an apoptosis inhibitor.

23. The method of claim 22 wherein said amiodarone product is an amiodarone analog, derivative or metabolite.

24. A method of reducing adverse effects resulting from administration of an amiodarone product to a subject comprising administering to said subject an antagonist of the renin-angiotensin-aldosterone system or an apoptosis inhibitor in an amount effective to reduce adverse effects of amiodarone product administration.

25. The method of claim 24 wherein said amiodarone product is an amiodarone analog, derivative or metabolite.

26. The method of claim 20, 22 or 24 wherein an antagonist of the renin-angiotensin-aldosterone system is administered.

27. The method of claim 20, 22 or 24 wherein an apoptosis inhibitor is administered.

28. The method of claim 26 wherein said antagonist of the renin-angiotensin-aldosterone system is an AT1 receptor antagonist.

29. The method of claim 28 wherein said AT1 receptor antagonist is selected from the group consisting of saralasin, losartan and L158809.

30. The method of claim 26 wherein said antagonist of the renin-angiotensin-aldosterone system is an ACE inhibitor.

31. The method of claim 30 wherein said ACE inhibitor is selected from the group consisting of captopril, lisinopril, ramipril and enalopril.

32. The method of claim 27 wherein said apoptosis inhibitor is a caspase inhibitor.

33. The method of claim 32 wherein said caspase inhibitor is selected from the group consisting of ZVAD-fmk, ZVAD-cmk, DEVD-fmk, DEVD-cmk, YVAD-fmk and YVAD-cmk.

34. The method of claim 27 wherein said apoptosis inhibitor is an endonuclease inhibitor.

35. The method of claim 21, 23 or 25 wherein said amiodarone product is selected from the group consisting of amiodarone, dronedarone and N-desethylamiodarone.


SEQUENCE LISTING (30 sequences)

SEQ ID NO: 1 - DNA, 24 bp, Artificial sequence
  Synthetic Primer
actggctgacttatgctttttact

SEQ ID NO: 2 - DNA, 25 bp, Artificial sequence
  Synthetic Primer
agaaaaggaaacaggaaacccagta

SEQ ID NO: 3 - DNA, 25 bp, Artificial sequence
  Synthetic Primer
ccttttggctactcttcctctatgg

SEQ ID NO: 4 - DNA, 25 bp, Artificial sequence
  Synthetic Primer
ttggtcacgggttatcctgttcttc

SEQ ID NO: 5 - DNA, 20 bp, Artificial sequence
  Synthetic Primer
agtacaacaagatcctgttg

SEQ ID NO: 6 - DNA, 24 bp, Artificial sequence
  Synthetic Primer
gatgtggccatcacattcgtcaga

SEQ ID NO: 7 - DNA, 20 bp, Artificial sequence
  Synthetic Primer
gagaggattcgtggcttgag

SEQ ID NO: 8 - DNA, 20 bp, Artificial sequence
  Synthetic Primer
gagacacgtgagaaggaaca

SEQ ID NO: 9 - DNA, 23 bp, Artificial sequence
  Synthetic Primer
atgaaggacaacttcagttttgc

SEQ ID NO: 10 - DNA, 23 bp, Artificial sequence
  Synthetic Primer
caaggggaactacataagatggc

SEQ ID NO: 11 - DNA, 20 bp, Artificial sequence
  Synthetic Primer
gtcagcttcatcatccagtt

SEQ ID NO: 12 - DNA, 20 bp, Artificial sequence
  Synthetic Primer
aggaagagcagcagccactg

SEQ ID NO: 13 - DNA, 20 bp, Artificial sequence
  Synthetic Primer
gctttcaacacctacgtcca

SEQ ID NO: 14 - DNA, 20 bp, Artificial sequence
  Synthetic Primer
agctgttgggtagactctgt

SEQ ID NO: 15 - DNA, 18 bp, Artificial sequence
  Synthetic Primer
ttctccctgctggccgag

SEQ ID NO: 16 - DNA, 18 bp, Artificial sequence
  Synthetic Primer
gggctctctctcatccgc

SEQ ID NO: 17 - DNA, 23 bp, Artificial sequence
  Synthetic Primer
aggccaaccgcgagaagatgacc

SEQ ID NO: 18 - DNA, 20 bp, Artificial sequence
  Synthetic Primer
gaagtccagggcgacgtagc

SEQ ID NO: 19 - DNA, 20 bp, Artificial sequence
  Synthetic Primer
cctcgctctctggacttatc

SEQ ID NO: 20 - DNA, 20 bp, Artificial sequence
  Synthetic Primer
cagacactgaggtgctgttg

SEQ ID NO: 21 - DNA, 25 bp, Artificial sequence
  Synthetic Primer
ctccccaaattcaagtgtactctcg

SEQ ID NO: 22 - DNA, 25 bp, Artificial sequence
  Synthetic Primer
gagtgacgtgtttaactctgcaagc

SEQ ID NO: 23 - DNA, 18 bp, Artificial sequence
  Synthetic Primer
ccgtgggagtcatcacgg

SEQ ID NO: 24 - DNA, 18 bp, Artificial sequence
  Synthetic Primer
cagggatctctggcggac

SEQ ID NO: 25 - DNA, 24 bp, Artificial sequence
  Synthetic Primer
agggtgggggaggtgctgaacagc

SEQ ID NO: 26 - DNA, 24 bp, Artificial sequence
  Synthetic Primer
gatgggggtgggggaccgtagcaa

SEQ ID NO: 27 - protein, 4 aa, Artificial Sequence
  Peptide substrate
YVAD

SEQ ID NO: 28 - protein, 4 aa, Artificial sequence
  Peptide substrate
DEVA

SEQ ID NO: 29 - protein, 4 aa, Artificial sequence
  Peptide substrate
DEVD

SEQ ID NO: 30 - protein, 4 aa, Artificial sequence
  Peptide substrate
YVAD